(19) United States
(12) Patent Application Publication
(10) Pub. No.: US 2010/0081126 A1
(43) Pub. Date: Apr. 1, 2010

(54) PROCESS FOR DESIGNING INHIBITORS OF INFLUENZA VIRUS STRUCTURAL PROTEIN-1

(75) Inventors: Gaetano T. Montelione, Highland Park, NJ (US); Robert M. Krug, Austin, TX (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: RUTGERS, THE STATE UNIVERSITY OF NEW JERSEY, New Brunswick, NJ (US)

(21) Appl. No.: 12/557,927

(22) Filed: Sep. 11, 2009

Related U.S. Application Data
(63) Continuation of application No. 10/534,782, filed on Nov. 16, 2007, now abandoned.
(60) Provisional application No. 60/477,453, filed on Jun. 10, 2003.
(60) Provisional application No. 60/425,661, filed on Nov. 13, 2002.

(51) Int. Cl. C12Q 1/70

(52) U.S. Cl. 435/5

(57) ABSTRACT

Disclosed are methods and compositions useful in identifying inhibitors of influenza virus, such as influenza A and B virus. Also disclosed are methods for preparing compositions for administration to animals, including humans infected with or to protect against influenza virus.

### CROSS-REFERENCE TO RELATED APPLICATIONS

This application is a continuation of U.S. application Ser. No. 10/534,782 filed on Nov. 16, 2007 as a national stage of PCT application PCT/US03/36292 filed on Nov. 13, 2003, which claims priority to provisional applications: 60/425,661 filed Nov. 13, 2002; and 60/477,453 filed Jun. 10, 2003, the contents of which are incorporated herein by reference.

### GOVERNMENT SUPPORT

Funding for research was partially supported by The National Institutes of Health under Contract Nos. GM47014 and AI11772.

### BACKGROUND ART

Influenza virus is a major human health problem. It causes a highly contagious acute respiratory illness known as influenza. The 1918-1919 pandemic of the “Spanish influenza” was estimated to cause about 500 million cases resulting in 20 million deaths worldwide (Robbins, 1986). The genetic determinants of the virulence of the 1918 virus have still not been identified, nor have the specific clinical preventatives or treatments that would be effective against such a re-emergence. See, Tumpey, et al., PNAS USA 99(15):13849-54 (2002). Not surprisingly, there is significant concern of the potential impact of a re-emergent 1918 or 1918-like influenza virus, whether via natural causes or as a result of bioterrorism. Even in nonpandemic years, influenza virus infection causes some 20,000-30,000 deaths per year in the United States alone (Wright & Webster, (2001) Orthomyxoviruses. In “Fields Virology, 4th Edition” (D. M. Knipe, and P. M. Howley, Eds.) pp. 1533-1579. Lippincott Williams & Wilkins, Philadelphia, Pa.). In addition, there are countless losses both in productivity and quality of life for people who overcome mild cases of the disease in just a few days or weeks. Another complicating factor is that influenza A virus undergoes continual antigenic change resulting in the isolation of new strains each year. Plainly, there is a continuing need for new classes of influenza antiviral agents.

Influenza viruses are the only members of the orthomyxoviridae family, and are classified into three distinct types (A, B, and C), based on antigenic differences between their nucleoprotein (NP) and matrix (M) protein (Pereira, (1969) Progr. Molec. Virol. 11:46). The orthomyxoviruses are enveloped animal viruses of approximately 100 nm in diameter. The influenza virions consist of an internal ribonucleoprotein core (a helical nucleocapsid) containing a single-stranded RNA genome, and an outer lipoprotein envelope lined inside by a matrix protein (M). The segmented genome of influenza A virus consists of eight molecules (seven for influenza C virus) of linear, negative polarity, single-stranded RNAs which encode ten polypeptides, including: the RNA-directed RNA polymerase proteins (PB2, PB1 and PA) and nucleoprotein (NP) which form the nucleocapsid; the matrix proteins (M1, M2); two surface glycoproteins which project from the lipoprotein envelope: hemagglutinin (HA) and neuraminidase (NA); and nonstructural proteins whose function is elucidated below (NS1 and NS2). Transcription and replication of the genome takes place in the nucleus and assembly occurs via budding on the plasma membrane. The viruses can reassort genes during mixed infections.

Replication and transcription of influenza virus RNA requires four virus-encoded proteins: the NP and the three components of the viral RNA-dependent RNA polymerase, PB1, PB2 and PA (Huang, et al., 1990, J. Virol. 64: 5669-5673). The NP is the major structural component of the virion, which interacts with genomic RNA, and is required for anti-termination during RNA synthesis (Beaton & Krug, 1986, Proc. Natl. Acad. Sci. USA 83:6282-6286). NP is also required for elongation of RNA chains (Shapiro & Krug, 1988, J. Virol. 62: 2285-2290) but not for initiation (Honda, et al., 1988, J. Biochem. 104: 1021-1026).

Influenza virus adsorbs via HA to sialyloligosaccharides in cell membrane glycoproteins and glycolipids. Following endocytosis of the virion, a conformational change in the HA molecule occurs within the cellular endosome which facilitates membrane fusion, thus triggering uncoating. The nucleocapsid migrates to the nucleus where viral mRNA is transcribed as the essential initial event in infection. Viral mRNA is transcribed by a unique mechanism in which viral endonuclease cleaves the capped 5′-terminus from cellular heterologous mRNAs which then serve as primers for transcription of viral RNA templates by the viral transcriptase. Transcripts terminate at sites 15 to 22 bases from the ends of their templates, where oligo(U) sequences act as signals for the template-independent addition of poly(A) tracts. Of the eight viral mRNA molecules so produced, six are monocistronic messages that are translated directly into the proteins representing HA, NA, NP and the viral polymerase proteins, PB2, PB1 and PA. (Influenza viruses have been isolated from humans, mammals and birds, and are classified according to their surface glycoproteins, hemagglutinin (HA) and neuraminidase (NA).)

The other two transcripts undergo splicing, each yielding two mRNAs, which are translated in different reading frames to produce M1, M2, non-structural protein-1 (NS1) and non-structural protein-2 (NS2). Eukaryotic cells defend against viral infection by producing a battery of proteins, among them interferons. The NS1 protein facilitates replication and infection of influenza virus by inhibiting interferon production in the host cell. The NS1 protein of influenza A virus is variable in length (Parvin et al., (1983) Virology 128:512-517) and is able to tolerate large deletions in the carboxyl terminus without affecting its functional integrity (Norton et al., (1987) 156(2):204-213). The NS1 protein contains two functional domains, namely a domain that binds double-stranded RNA (dsRNA), and an effector domain. The effector domain is located in the C-terminal domain of the protein. Its functions are relatively well established. Specifically, the effector domain functions by interacting with host nuclear proteins to carry out the nuclear RNA export function. (Qian et al., (1994) J. Virol. 68(4):2433-2441).

The dsRNA-binding domain of the NS1A protein is located at its amino terminal end (Qian et al., 1994). An amino-terminal fragment, which is comprised of the first 73 amino-terminal amino acids [NS1A(1-73)], possesses all the dsRNA-binding properties of the full-length protein (Qian et al, (1995) RNA 1:948-956). NMR solution and X-ray crystal structures of NS1A(1-73) have shown that in solution it forms a symmetric homodimer with a unique six-helical chain fold (Chien et al., (1997) Nature Struct. Biol.

4:891-895; Liu et al., (1997) Nature Struct. Biol. 4:896-899). Each polypeptide chain of the NS1A(1-73) domain consists of three alpha-helices corresponding to the segments Asn⁴-Asp²⁴ (helix 1), Pro³¹-Leu⁵⁰ (helix 2), and Ile⁵⁴-Lys⁷⁰ (helix 3). Preliminary analysis of NS1A(1-73) surface features suggested two possible nucleic acid binding sites, one involving the solvent exposed stretches of helices 2 and 2′ comprised largely of the basic side chains, and the other at the opposite side of the molecule that includes some lysine residues of helices 3 and 3′ (Chien et al., 1997). Subsequent sited-directed mutagenesis experiments indicated that the side chains of two basic amino acids (Are and Lys⁴¹) in the second alpha-helix are the only amino acid side chains that are required for the dsRNA binding activity of the intact dimeric protein (Wang et al., 1999 RNA 5:195-205). These studies also demonstrated that dimerization of the NS1A(1-73) domain is required for dsRNA binding. However, aside from binding dsRNA (e.g., Hatada & Futada, (1992) J. Gen. Virol., vol. 73(12):3325-3329; Lu et al., (1995) Virology, 214:222-228; Wang et al., (1999)), the precise function of the dsRNA binding domain has not been established.

### SUMMARY OF THE INVENTION

The present invention exploits Applicants' discoveries regarding exactly how the NS1 protein, and particularly the dsRNA binding domain in the N-terminal portion of the protein participate in the infectious process of influenza virus. Applicants have discovered that the RNA-binding domain of the NS1A protein is critical to the replication and pathogenicity of influenza A virus. Applicants have discovered that when the binding domain of NS1A binds dsRNA in the host cell, the cell is unable to activate portions of its anti-viral defense system that inhibit production of viral protein. dsRNA binding by NS1A causes the enzyme, double-stranded-RNA-activated protein kinase (“PKR”) to remain inactivated such that it cannot catalyze the phosphorylation of translation initiation factor eIF2α, which would otherwise be able to inhibit viral protein synthesis and replication. Previous reports by others indicated that the amino acids involved in inhibition of PKR do not include those that are required for dsRNA binding. Contrary to these reports, Applicants have also discovered that two amino acid residues in the NS1 protein for both influenza A and B viruses (i.e., NS1A: arginine 38 (R³⁸), and lysine 41 (K⁴¹); NS1B: arginine) (R⁵⁰), and arginine 53 (R⁵³)) that are key residues in terms of RNA binding are also involved in the ability of the dsRNA binding domain to disarm the host cell in this manner. Applicants have discovered the structural interface of NS1A or NS1B with dsRNA, and defined structural features of this interface which, based on the above, are targets for drug design. Applicants have invented a set of assays for characterizing interactions between NS1A or NS1B, and dsRNA, which can be used in small scale and/or high-throughput screening for inhibitors of this interaction. Applicants have also discovered that an amino-terminal fragment, which is comprised of the first 93 amino-terminal amino acids [NS1B(1-93)], possesses all the dsRNA-binding properties of the full-length NS1 protein of influenza B virus.

One aspect of the present invention is directed to a method of identifying compounds having inhibitory activity against an influenza virus, comprising:

a) preparing a reaction system comprising an NS1 protein of an influenza virus or a dsRNA binding domain thereof, a dsRNA that binds said protein or binding domain thereof, and a candidate compound; and

b) detecting extent of binding between the NS1 protein and the dsRNA, wherein reduced binding between the NS1 protein and the dsRNA in the presence of the compound relative to a control is indicative of inhibitory activity of the compound against the influenza virus. The compounds identified as having inhibitory activity against influenza virus can then be further tested to determine whether they would be suitable as drugs. In this way, the most effective inhibitors of influenza virus replication can be identified for use in subsequent animal experiments, as well as for treatment (prophylactic or otherwise) of influenza virus infection in animals including humans.

Accordingly, another aspect of the present invention is directed to a method of identifying compounds having inhibitory activity against an influenza virus, comprising:

a) preparing a reaction system comprising an NS1 protein of an influenza virus or a dsRNA binding domain thereof, a dsRNA that binds said protein or binding domain thereof, and a candidate compound;

b) detecting extent of binding between the NS1 protein and the dsRNA, wherein reduced binding between the NS1 protein and the dsRNA in the presence of the compound relative to a control is indicative of inhibitory activity of the compound against the influenza virus; and

c) determining extent of a compound identified in b) as having inhibitory activity to inhibit growth of influenza virus in vitro.

In some embodiments, the method further entails d) determining extent of a compound identified in c) as inhibiting growth of influenza virus in vitro, to inhibit replication of influenza virus in a non-human animal.

A further aspect of the present invention is directed to a method of preparing a composition for inhibiting replication of influenza virus in vitro or in vivo, comprising:

a) preparing a reaction system comprising an NS1 protein of an influenza virus or a dsRNA binding domain thereof, a dsRNA that binds said protein or binding domain thereof, and a candidate compound;

b) detecting extent of binding between the NS1 protein and the dsRNA, wherein reduced binding between the NS1 protein and the dsRNA in the presence of the compound relative to a control is indicative of inhibitory activity of the compound against the influenza virus;

c) determining extent of a compound identified in b) as having inhibitory activity to inhibit growth of influenza virus in vitro;

d) determining extent of a compound identified in c) as inhibiting growth of influenza virus in vitro, to inhibit replication of influenza virus in a non-human animal; and

e) preparing the composition by formulating a compound identified in d) as inhibiting replication of influenza virus in a non-human animal, in an inhibitory effective amount, with a carrier.

In each of the above aspects of the present invention, some embodiments entail labeling the NS1 protein or the dsRNA with a fluorescent molecule, and then determining extent of binding via fluorescent resonance energy transfer or fluorescence polarization. In other embodiments, the control is extent of binding between the dsRNA and the NS1 protein or a dsRNA binding domain that lacks amino acid residues R³⁸ and/or K⁴¹. Other embodiments entail methods of assaying for influenza virus NS1 protein/dsRNA complex formation. Yet still other embodiments entail methods of using a influenza virus NS1 protein/dsRNA complex formation in screening for or optimizing inhibitors. These embodiments include NMR chemical shift perturbation of the NS1 protein or RNA gel filtration sedimentation equilibrium and virtual screening using the structure of NS1 protein and the model of the NS1-RNA complex

A further aspect of the present invention is directed to a composition comprising a reaction mixture comprising a complex of an NS1 protein of influenza virus, or a dsRNA binding fragment thereof, and a dsRNA that binds said protein. In some embodiments, the NS1 protein is an NS1A protein, or the dsRNA binding fragment thereof, the 73 N-terminal amino acid residues of the protein. In other embodiments, the NS1 protein is an NS1B protein, or the dsRNA binding fragment thereof, the 93 N-terminal amino acid residues of the protein. In other embodiments, the composition further contains a candidate or test compound being tested for inhibitory activity against influenza virus.

A still further aspect of the present invention is directed to a method of identifying a compound that can be used to treat influenza virus infections comprising using the structure of a NS1 protein or a dsRNA binding domain thereof, NS1A(1-73) or NS1B(1-93), and the three dimensional coordinates of a model of the NS1-RNA complex in a drug screening assay.

These and other aspects of the present invention will be better appreciated by reference to the following drawings and detailed description.

The file of this patent contains at least one drawing executed in color. Copies of this patent with color drawing(s) will be provided by the Patent and Trademark Office upon request and payment of the necessary fee.

### BRIEF DESCRIPTION OF THE DRAWINGS

FIG. 1. Gel shift assay for different duplexes on their ability to bind NS1A(1-73). This experiment was performed under standard conditions using indicated ³²P-labeled double-stranded nucleic acids (1.0 nM) and either with (+); or without (−) 0.4 μM NS1A(1-73).

FIG. 2. Gel filtration chromatography profiles of different duplexes in the presence of NS1A(1-73): (A) dsRNA; (B) RNA-DNA hybrid; (C) DNA-RNA hybrid; (D) dsDNA. The major peaks between 20 and 30 min correspond to the duplexes, except for the first peak in (A) which is from the NS1A(1-73)-dsRNA complex.

FIG. 3. Gel filtration chromatograms of the purified NS1A(1-73)-dsRNA complex. (A) 4 μM, 100 μl of the fresh complex sample; (B) 4 μM, 100 μl of the complex sample after one month.

FIG. 4. (A) Determination of the stoichiometry based on sedimentation equilibrium at 16000 rpm on three samples with loading concentrations of 0.6 ( ) 0.3(Δ) and 0.5 (not shown, to avoid the overlap of data points) absorbance unit. The solid line is the joint fit of the three sets of data assuming a 1:1 stoichiometry of the dsRNA:NS1 complex; the insert shows the random residual plots of the fit. The dotted line is drawn assuming a 1:2 stoichiometry of the dsRNA:NS1 complex. (The 2:1 complex has nearly identical concentration distribution profile as those shown by the dotted lines because of the nearly identical reduced molecular weight of dsRNA and NS1 protein (see infra). (B): Estimation of the dissociation constant from sedimentation equilibrium of three samples (see above) at speed 16000 ( ) 22000 (◯) and 38000 (Δ) rpm. Only the data of the sample with loading concentration of 0.5 absorbance unit is shown here. The solid lines are the global fit using an ideal monomer-dimer model of NONLIN, and the dissociation constant is calculated from the fitting results using Eq. 7. The insert shows the residual plots of the fit.

FIG. 5. (A) Two-dimensional ¹H-¹⁵N HSQC spectrum of 2.0 mM uniformly ¹⁵N-enriched NS1A(1-73) at 20° C., pH 6.0 in 95% H₂O/5% D₂O containing 50 mM ammonium acetate and 1 mM sodium azide. The cross peaks are labeled with respective resonance assignments indicated by the one-letter code of amino acids and a sequence number. Also shown are side-chain NH resonance of the tryptophan and side-chain NH₂ resonances for glutamines and asparagines. The peaks assigned to N^{ε}-H^{ε} resonances of arginines are folded in the F1 (¹⁵N) dimension from their positions further upfield. (B) An overlay of represented ¹H^{N}-¹⁵N HSQC spectra for ¹⁵N-enriched NS1A(1-73) uncomplexed (red) and complexed (blue) with 16-bp dsRNA at pH 6.0, 20° C. Labels correspond to amide backbone assignments of well-resolved cross peaks of the free protein.

FIG. 6. (A) Ribbon diagram of NS1A(1-73) showing the results of chemical shift perturbation measurements. Residues of NS1A(1-73) which give shift perturbations in NMR spectra of the NS1A(1-73)-dsRNA complex are colored in cyan, residues that are not changed in the chemical shifts of their amide ¹⁵N and ¹H are colored in pink, and white represents the chemical shift assignments of the residues that cannot be identified in 2D HSQC spectra due to the overlapped cross peaks. (B) Side chains shown in FIG. 6B are also displayed here with all the basic residues labeled. Note that the binding epitope of NS1A(1-73) to dsRNA appears to be on the bottom of this structure.

FIG. 7. CD spectra of the purified NS1A(1-73)-dsRNA complex (A), and the mixtures of duplexes and NS1A(1-73): RNA-DNA hybrid (B), and DNA-RNA hybrid (C). Orange: experimental CD spectra of the mixtures (1:1 molar ratio of duplex and protein dimer). Red: duplex alone. Blue: NS1A(1-73) alone. Green: calculated sum spectra of duplex and NS1A(1-73).

FIG. 8. A model of the dsRNA binding properties of NS1A(1-73). The model is useful for the purpose of designing experiments to test the implied hypotheses. Phosphate backbones and base-pairs of dsRNA are shown in orange and yellow, respectively. All side chains of Arg and Lys residues are labeled in green.

### BEST MODE OF CARRYING OUT THE INVENTION

The present invention provides methods of designing specific inhibitors of dsRNA binding domains of NS1 proteins from both influenza A and B viruses. The amino acid sequences of the dsRNA binding domains of NS1 proteins of influenza A, particularly the R³⁸ and K⁴¹ amino acid residues, are substantially conserved. Multiple sequence alignments for the NS1 protein of various strains of influenza A virus is described in Table 1.

In addition, by way of example only, the amino acid sequence of the NS1 protein of various strains of influenza A virus is set forth below.

The amino acid sequence of the NS1 protein of Influenza A virus, A/Udorn/72 (SEQ ID NO: 1):

```
    1 MDPNTVSSFQ VDCFLWHVRK RVADQELGDA PFLDRLRRDQ KSLRGRGSTL GLDIETATRA
  61 GKQIVERILK EESDEALKMT MASVPASRYL TDMTLEEMSR EWSMLIPKQK VAGPLCIRMD
121 QAIMDKNIIL KANFSVIFDR LETLILLRAF TEEGAIVGEI SPLPSLPGHT AEDVKNAVGV
181 LIGGLEWNDN TVRVSETLQR FAWRSSNENG RPPLTPKQKR EMAGTIRSEV
```

The amino acid sequence of the NS1 protein of Influenza A virus, A/goose/Guangdong/3/1997 (H5N1) (SEQ ID NO: 2):

```
    1 MDSNTITSFQ VDCYLWHIRK LLSMSDMCDA PFDDRLRRDQ KALKGRGSTL GLDLRVATME
  61 GKKIVEDILK SETNENLKIA IASSPAPRYV TDMSIEEMSR EWYMLMPRQK ITGGLMVKMD
121 QAIMDKRIIL KANFSVLFDQ LETLVSLRAF TESGAIVAEI SPIPSVPGHS TEDVKNAIGI
181 LIGGLEWNDN SIRASENIQR FAWGIRDENG GPSLPPKQKR YMAKRVESEV
```

The amino acid sequence of the NS1 protein of Influenza A VIRUS A/QUAIL/NANCHANG/12-340/2000 (H1N1) (SEQ ID NO: 3):

```
    1 ELGDAPFLDR LRRDQKSLKG RGSTLGLNIE TATCVGKQIV ERILKEESDE AFKMTMASAL
  61 ASRYLTDMTI EEMSRDWFML MPKQKVAGPL CVRMDQAIMD KNIILKANFS VIFDRLETLT
121 LLRAFTEEGA IVGEISPLPS LPGHTNEDVK NAIGVLIGGL EWNDNTVRVS ETL
```

The amino acid sequence of the NS1 protein of Influenza A virus gi|577477|gb|AAA56812.1|[577477] (SEQ ID NO: 4):

```
    1 MDSNTVSSFQ VDCFLWHVRK RFADQEMGDA PFLDRLRRDQ KSLGGRGSTL GLDIETATRA
  61 GKQIVEPILE EESDEALKMT IASAPVSRYL PDMTLEEMSR DWFMLMPKQK VAGSLCIRMD
121 QAIMDKNITL KANFSIIFDR LETLILLRAF TEEGAIVGEI SPVPSLPGHT DEDVKNAIGV
181 LIGGLEWNDN TVRDSETLQR FAWRSSNEDR RPPLPPKQKR KMARTIESEV
```

The amino acid sequence of the NS1 protein of Influenza A virus gi|413859|gb|AAA43491.1|[413859] (SEQ ID NO: 5):

```
    1 MDSNTVSSFQ VDCFLWHVRK RFADQERGDA PFLDRLRRDQ KSLRGRGSTL GLDIETATCA
  61 GKQIVERILK EESDEALKMT IASVPASRYL TDMTLEEMSR DWFMLMPKQK VAGSLCIRMD
121 QAIMDKNIIL KANFSVIFDR LETLILLRAF TEEGAIVGEI SPLPSLPGHT DEDVKNAIGV
181 LIGGLEWNDN TVRVSETLQR FAWRSSNEDG RPPFPPKQKR KMARTIESEV
```

The amino acid sequence of the NS1 protein of Influenza A virus gi|325085|gb|AAA43684.1|[325085] (SEQ ID NO: 6):

```
    1 MDSNTVSSFQ VDCFLWHVRK RFADQKLGDA PFLDRLRRDQ KSLRGRASTL GLDIETATRA
  61 GKQIVERILE EESNEALKMT IASVPASRYL TDMTLEEMSR DWFMLMPKQK VAGSLCIRMD
121 QAIMEKSIIL KANFSVIFDR LETLILLRAF TEEGAIVGEI SPLHSLPGHT DEDVKNAVGV
181 LIGGLEWNGN TVRVSENLQR FAWRSRNENE RPSLPPKQKR EVAGTIRSEV
```

The amino acid sequence of the NS1 protein of Influenza A virus gi|324876|gb|AAA43572.1|[324876] (SEQ ID NO: 7):

```
    1 NTVSSFQVDC FLWHVRKRFA DQELGDAPFL DRLRRDQKSL RGRGSTLGLD IETATRAGKQ
  61 IVERILVEES DEALKMTIVS MPASRYLTDM TLEEMSRDWF MLMPKQKVAG SLCIRMDQAI
121 MDKNIILKAN FSVISDRLET LILLRAFTEE GAIVGEISPL PSLPGHTDED VKNAIGDLIG
181 GLEWNDNTVR VSETLQRFAW RSSNEDGRPL LPPKQKRKMA RTIESEV
```

The amino acid sequence of the NS1 protein of Influenza A virus gi|324862|gb|AAA43553.1|[324862] (SEQ ID NO: 8):

```
    1 MDPNTVSSFQ VDCFLWHVRK QVADQELGDA PFLDRLRRDQ KSLRGRGSTL GLNIETATRV
  61 GKQIVERILK EESDEALKMT MASAPASRYL TDMTIEEMSR DWFMLMPKQK VAGPLCIRMD
121 QAIMDKNIIL KANFSVIFDR LETLILLRAF TEAGAIVGEI SPLPSLPGHT NEDVKNAIGV
181 LIGGLEWNDN TVRVSKTLQR FAWRSSDENG RPPLTPK
```

The amino acid sequence of the NS1 protein of Influenza A virus gi|324855|gb|AAA43548.1|[324855] (SEQ ID NO: 9):

```
    1 NTVSSFQVDC FLWHVLKRFA DQELGDAPFL DRLRRDQKSL RGRGSTLGLD IETATRAGKQ
  61 IVERILEEES DEALKMNIAS VPASRYLTDM TLEEMSRDWF MLMPKQKVAG SLCIRMDQAI
121 MDKNIILKAN FSVIFDRLET LILLRAFTEE GAIVGEISPL PSLPGHTDED VKNAIGILIG
181 GLEWNDNTVR VSETLQRFAW RSSNEDGRPP LPPKQKWKMA RTIEPEV
```

The amino acid sequence of the NS1 protein of Influenza A virus gi|324778|gb|AAA43504.1|[324778] (SEQ ID NO: 10):

```
    1 NTVSSFQVDC FLWHVRKRFA DLELGDAPFL DRLCRDQKSL RGRSSTLGLD IETATRAGKQ
  61 IVERILEEES DETLKMTIAS APAFRYPTDM TLEEMSRDWF MLMPKQKVAG SLCIRMDQAI
121 MDKNIILKAN FSVIFDRLET LILLRAFTEE GAIVGEISPL PSLPGHTNED VKNAIGDLIG
181 GLEWNDNTVR VSETLQRFAW RSSNEGGRPP LPPKQKRKMA RTIESEV
```

The amino acid sequence of the NS1 protein of Influenza A virus, A/PR/8/34 (SEQ ID NO: 11):

```
    1 MDSNTITSFQ VDCYLWHIRK LLSMRDMCDA PFDDRLRRDQ KALKGRGSTL GLDLRVATME
  61 GKKIVEDILK SETDENLKIA IASSPAPRYI TDMSIEEISR EWYMLMPRQK ITGGLMVKMD
121 QAIMDKRITL KANFSVLFDQ LETLVSLRAF TDDGAIVAEI SPIPSMPGHS TEDVKNAIGI
181 LIGGLEWNDN SIRASENIQR FAWGIRDENG GPPLPPKQKR YMARRVESEV
```

The amino acid sequence of the NS1 protein of Influenza A virus, A/turkey/Oregon/71 (H7N5) (SEQ ID NO: 12):

```
    1 MDSNTITSFQ VDCYLWHIRK LLSMRDMCDA PFDDRLRRDQ KALKGRGSTL GLDLRVATME
  61 GKKIVEDILK SETDENLKIA IASSPAPRYI TDMSIEEISR EWYMLMPRQK ITGGLMVRPL
121 WTRG
```

The amino acid sequence of the NS1 protein of Influenza A virus, A/Hong Kong/1073/99(H9N2) (SEQ ID NO: 13):

```
    1 MDSNTVSSFQ VDCFLWHVRK RFADQELGDA PFLDRLRRDQ KSLRGRGSTL GLDIRTATRE
  61 GKHIVERILE EESDEALKMT IASVPASRYL TEMTLEEMSR DWLMLIPKQK VTGPLCIRMD
121 QAVMGKTIIL KANFSVIFNR LEALILLRAF TDEGAIVGEI SPLPSLPGHT DEDVKNAIGV
181 LIGGLEWNDN TVRVSETLQR FTWRSSDENG RSPLPPKQKR KVERTIEPEV
```

The amino acid sequence of the NS1 protein of Influenza A virus, A/Fort Monmouth/1/47-MA(H1N1) (SEQ ID NO: 14):

```
    1 MDPNTVSSFQ VDCFLWHVRK RVADQELGDA PFLDRLRRDQ KSLKGRGSTL GLNIETATRV
  61 GKQIVERILK EESDEALKMT MASAPASRYL TDMTIEEMSR DWFMLMPKQK VAGPLCIRMD
121 QAIMDKSIIL KANFSVIFDR LETLILLRAF TEEGAIVGEI SPLPSLPGHT NEDVKNAIGV
181 LIGGLEWNDN TVRVSKTLQR FA
```

Strains of influenza B virus also possess similar dsRNA binding domains. Multiple sequence alignments for the NS1 protein of various strains of influenza B virus are described in Table 2.

In addition, by way of example only, the amino acid sequence of the NS1 protein of various strains of influenza B virus is set forth below.

The amino acid sequence of the NS1 protein of the influenza B virus (B/Lee/40) (SEQ ID NO: 15):

```
    1 MADNMTTTQI EVGPGATNAT INFEAGILEC YERFSWQRAL DYPGQDRLHR LKRKLESRIK
  61 THNKSEPENK RMSLEERKAI GVKMMKVLLF MDPSAGIEGF EPYCVKNPST SKCPNYDWTD
121 YPPTPGKYLD DIEEEPENVD HPIEVVLRDM NNKDARQKIK DEVNTQKEGK FRLTIKRDIR
181 NVLSLRVLVN GTFLKHPNGD KSLSTLHRLN AYDQNGGLVA KLVATDDRTV EDEKDGHRIL
241 NSLFERFDEG HSKPIRAAET AVGVLSQFGQ EHRLSPEEGD N
```

The amino acid sequence of the NS1 protein of the influenza B virus B/Memphis/296 (SEQ ID NO: 16):

```
    1 MADNMTTTQI EVGPGATNAT INFEAGILEC YERLSWQRAL DYPGQDRLNR LKRKLESRIK
  61 THNKSEPESK RMSLEERKAI GVKMMKVLLF MDPSAGIEGF EPYCMKSSSN SNCPKYNWTD
121 YPSTPGRCLD DIEEEPEDVD GPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERLNEG HSKPIRAAET AVGVLSQFGQ EHRLSPEEGD N
```

The amino acid sequence of the NS1 protein of the influenza B virus gi|325264|gb|AAA43761.1|[325264] (SEQ ID NO: 17):

```
    1 MADNMTTTQI EVGPGATNAT INFEAGILEC YERLSWQRAL DYPGQDRLNR LKRKLESRIK
  61 THNKSEPESK RMSLEERKAI GVKMMKVLLF MNPSAGIEGF EPYCMKNSSN SNCPNCNWTD
121 YPPTSGKCLD DIEEEPENVD DPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERFNEG HSKPIRAAET AVGVLSQFGQ EHRLSPEEGD N
```

The amino acid sequence of the NS1 protein of the influenza B virus B/Ann Arbor/1/66 [gi|325261|gb|AAA43759.1|[325261]] (SEQ ID NO: 18):

```
    1 MADNMTTTQI EVGPGATNAT INFEAGILEC YERLSSQRAL DYPGQDRLNR LKRKLESRIK
  61 THNKSEPESK RMSLEERKAI GVKMMKVLLF MNPSAGIEGF EPYCMKNSSN SNCPNCNWTD
121 YPPTPGKCLD DIEEEPENVD DPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERFNEG HSKPIRAAET AVGVLSQFGQ EHRLSPEEGD N
```

The amino acid sequence of the NS1 protein of the influenza B virus gi|325256|gb|AAA43756.1|[325256] (SEQ ID NO: 19):

```
    1 MADNMTTTQI EVGPGATNAT INFEAGILEC YERFSWQRAL DYPGQDRLHR LKRKLESRIK
  61 THNKSEPENK RMSLEERKAI GVKMMKVLLF MDPSAGIEGF EPYCVKNPST SKCPNYDWTD
121 YPPTPGKYLD DIEEEPENVD HPIEVVLRDM NNKDARQKIK DEVNTQKEGK FRLTIKRDIR
181 NVLSLRVLVN GTFLKHPNGD KSLSTLHRLN AYDQNGGLVA KLVATDDRTV EDEKDGHRIL
241 NSLFERFDEG HSKPIRAAET AVGVLSQFGQ EHRLSPEEGD N
```

The amino acid sequence of the NS1 protein of the influenza B virus (B/Shangdong/7/97) (SEQ ID NO: 20):

```
    1 MADNMTTTQI EVGPGATNAT INFEAGILEC YERLSWQRAL DYPGQDRLNR LKRKLESRIK
  61 THNKSEPESK RMSLEERKAI GVKMMKVLLF MDPSAGIEGF EPYCMKSSSN SNYPKYNWTD
121 YPSTPGRCLD DIEEETEDVD DPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERLNEG HSKPIRAAET AVGVLSQFGQ EHRLSPEEGD N
```

The amino acid sequence of the NS1 protein of the influenza B virus (B/Nagoya/20/99) (SEQ ID NO: 21):

```
    1 MADNMTTTQI EVGPGATNAT INFEAGILEC YERLSWQRAL DYPGQDRLNR LKRKLESRIK
  61 THNKSEPESK RMSLEERKAI GVKMMKVLLF MDPSAGIEGF EPYCMKSSSN SNYPKYNWTN
121 YPSTPGRCLD DIEEETEDVD DPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERLNEG HPKPIRAAET AVGVLSQFGQ EHRLSPEEGD N
```

The amino acid sequence of the NS1 protein of the influenza B virus (B/Saga/S172/99) (SEQ ID NO: 22):

```
    1 MADNMTTTQI EVGPGATNAT INFEAGILEC YERLSWQRAL DYPGQDRLNR LKRKLESRIK
  61 THNKSEPESK RMSLEERKAI GVKMMKVLLF MDPSAGIEGF EPYCMKSSSN SNCPKYNWTD
121 YPSTPGRCLD DIEEEPEDVD GPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERLNEG HSKPIRAAET AVGVLSQFGQ EHRLSPEEGD N
```

The amino acid sequence of the NS1 protein of the influenza B virus (B/Kouchi/193/99) (SEQ ID NO: 23):

```
    1 MADNMTTTQI EVGPGATNAT INFEAGILEC YERLSWQRAL DYPGQDRLNR LKRKLESRIK
  61 THNKSEPESK RMSLEERKAI GVKMMKVLLF MDPSAGIEGF EPYCMKSSSN SNCPKYNWTD
121 YPSTPGRCLD DIEEEPEDVD GPTEIVLRDM NNKDARQKIK EEVNTQKEGK FRLTIKRDIR
181 NVLSLRVLVN GTFLKHPNGY KSLSTLHRLN AYDQSGRLVA KLVATDDLTV EDEEDGHRIL
241 NSLFERLNEG HSKPIRAAET AMGVLSQFGQ EHRLSPEEGD N
```

Thus, use in the disclosed inventions of any one NS1 protein or fragment thereof that binds dsRNA (and which has intact R³⁸, K⁴¹ residues for NS1A, and intact R⁵⁰, R⁵³ residues for NS1B) will serve to identify compounds having inhibitory activity against strains of influenza A virus, as well as strains of influenza B virus, respectively.

The present invention does not require that the proteins be naturally occurring. Analogs of the NS1 protein that are functionally equivalent in terms of possessing the dsRNA binding specificity of the naturally occurring protein, may also be used. Representative analogs include fragments of the protein, e.g., the dsRNA binding domain. Other than fragments of the NS1 protein, analogs may differ from the naturally occurring protein in terms of one or more amino acid substitutions, deletions or additions. For example, functionally equivalent amino acid residues may be substituted for residues within the sequence resulting in a change of sequence. Such substitutes may be selected from other members of the class to which the amino acid belongs; e.g., the nonpolar (hydrophobic) amino acids include alanine, leucine, isoleucine, valine, proline, phenylalanine, tryptophan, and methionine; the polar neutral amino acids include glycine, serine, threonine, cysteine, tyrosine, asparagine, and glutamine; the positively charged (basic) amino acids include arginine, lysine, and histidine; the negatively charged (acidic) amino acids include aspartic and glutamic acid. The R³⁸ and K⁴¹ residues for NS1A can be changed but there are limitations. For example, Applicants determined that replacing R³⁸ with a Lysine residue had no detectable effect on RNA binding whereas substitution with an alanine residue abolished this activity, indicating that a positively charged basic side chain at this position (i.e. either lysine or arginine) is required for these dsRNA-protein interactions; substitutions with any of the remaining 17 natural common amino acid residues are expected, like the alanine substitution, to abolish this activity. In preferred embodiments, however, the R³⁸ and K⁴¹ residues remain intact. The above-described statements are equally applicable to the R⁵⁰ and R⁵³ residues of NS1B. For purposes of the present invention, the term “dsRNA binding domain” is intended to include analogs of the NS1 protein that are functionally equivalent to the naturally occurring protein in terms of binding to dsRNA.

The NS1 proteins of the present invention may be prepared in accordance with established protocols. The NS1 protein of influenza virus, or a dsRNA binding domain thereof, may be derived from natural sources, e.g., purified from influenza virus infected cells and virus, respectively, using protein separation techniques well known in the art; produced by recombinant DNA technology using techniques known in the art (see e.g., Sambrook et al., 1989, Molecular Cloning: A Laboratory Manual, Cold Spring Harbor Laboratories Press, Cold Spring Harbor, N.Y.); and/or chemically synthesized in whole or in part using techniques known in the art; e.g., peptides can be synthesized by solid phase techniques, cleaved from the resin and purified by preparative high performance liquid chromatography (see, e.g., Creighton, 1983, Proteins: Structures and Molecular Principles, W. H. Freeman & Co., N.Y., pp. 50-60). Protocols for biosynthesis of the peptide defined by amino acid residues 1-73 of NS1A, with or without isotopic labeling suitable for NMR analysis, have been reported in Qian, et al., RNA 1(9):948-56 (1995) and Chien et al., (1997). The composition of the synthetic peptides may be confirmed by amino acid analysis or sequencing; e.g., using the Edman degradation procedure (see e.g., Creighton, 1983, supra at pp. 34-49).

Another discovery made by Applicants is that the NS1A(1-73) dsRNA-binding domain of influenza virus nonstructural protein 1 differs from the predominant class of dsRNA-binding domains, referred to as dsRBMs, that are found in a large number of eukaryotic and prokaryotic proteins. The proteins which contain the dsRBM domain include eukaryotic protein kinase R(PKR) (Nanduri et al., 1998), a kinase that plays a key role in the cellular antiviral response, Drosophila melonogaster Staufen (Ramos et al., 2000), and Escherichia coli Rnase III (Kharrat et al., 1995). The dsRBM domain comprises a monomeric α-β-β-β-α fold. Structural analysis has established that this domain spans two minor grooves and the intervening major groove of the dsRNA target (Ryter & Schultz, 1998). Several amino acids of the dsRBM domain are involved in direct and water-mediated interactions with the phosphodiester backbone, ribose 2′-OH groups, and a small number of bases. As a result of this binding, the canonical A-form dsRNA duplex is distorted upon complex formation. This binding is relatively strong, with a K_{d} of approximately 1 nmolar. Thus, the methods of the present invention exploit a phenomenon that occurs exclusively between a viral protein and dsRNA present in the infected eucaryotic cell. Therefore, compounds identified by the methods of the present invention might not otherwise affect normal cellular function.

Applicants' also discovered that one of the intracellular functions of the RNA-binding domain of the NS1A protein is to prevent the activation of PKR by binding dsRNA. Applicants generated recombinant A/Udorn/72 viruses that encode NS1A proteins whose only defect is in RNA binding. Because the R at position 38 (R³⁸) and K at position 41 (K⁴¹) are the only amino acids that are required solely for RNA binding, we substituted A for either one or both of these amino acids. The three mutant viruses are highly attenuated: the R³⁸ and K⁴¹ mutant viruses form pin-point plaques, and the double mutant (R38/K41) does not form visible plaques. During high multiplicity infection of A549 cells with any of these mutant viruses, PKR is activated, eIF2a is phosphorylated, and viral protein synthesis is inhibited. Surprisingly, after its activation, PKR is degraded. The R38/K41 double mutant is most effective in inducing PKR activation.

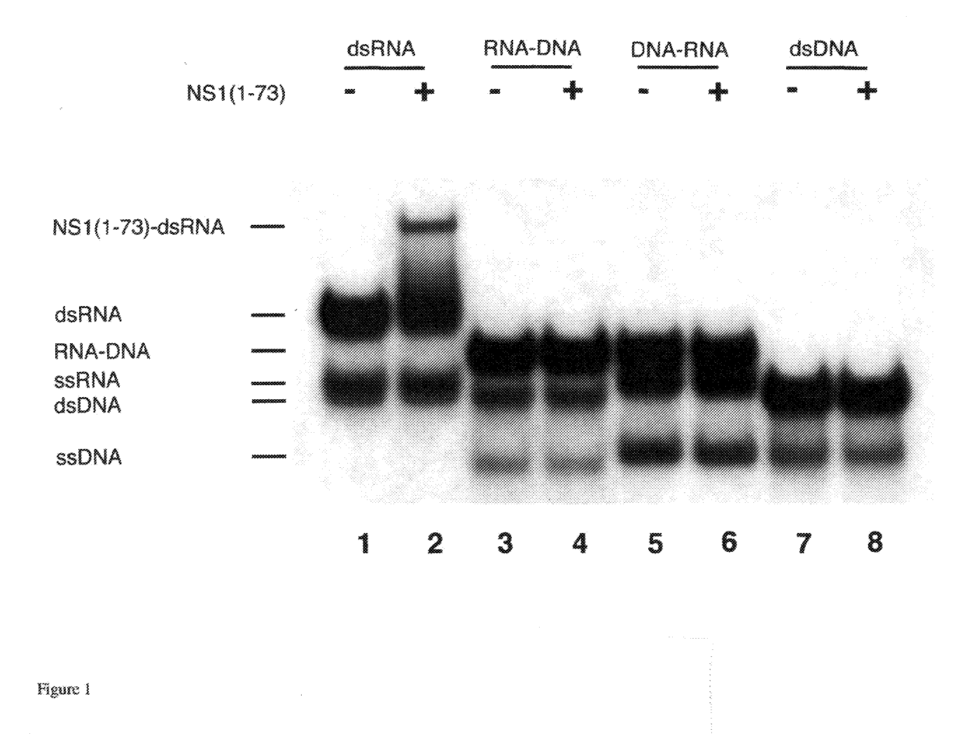

NS1A(1-73) binds dsRNA, but not dsDNA or RNA/DNA hybrids. NS1A(1-73) and the full length NS1A protein have been shown to bind double-stranded RNAs (dsRNAs) with no sequence specificity (Lu et al., (1995) Virology 214, 222-228, Qian et al., (1995) RNA 1, 948-956, Wang et al., 1999), but until the present invention, it had not been determined whether NS1A(1-73) or the NS1A protein bind RNA-DNA hybrids or dsDNA. Applicants incubated NS1A(1-73) with four ³²P-labeled duplexes: 16-bp dsRNA (RR), dsDNA (DD), and two RNA-DNA hybrid duplexes (RD and DR). These mixtures are then analyzed on a native 15% polyacrylamide gel (FIG. 1). As reported by others (Roberts and Crothers (1992) Science 258, 1463-1466; Ratmeyer et al., (1994) Biochemistry 33, 5298-5304; Lesnik and Freier (1995) Biochemistry 34, 10807-10815), Applicants observed the following migration pattern for the free duplexes on the native gel (fastest to slowest): DD>DR/RD>RR (lanes 1, 3, 5, and 7, respectively). More importantly, only dsRNA is found to form a complex with NS1A(1-73) producing a 30% gel shift (lane 2), whereas all the other duplexes fail to bind to the protein (lanes 4, 6, and 8). These data indicate that NS1A(1-73) specifically recognizes the conformational and/or structural features of dsRNA (A-form conformation) which are distinct from those of dsDNA (B-form conformation) or RNA/DNA hybrids (intermediate A/B conformations) under these conditions.

The length and ribonucleotide sequence of the dsRNA are not critical. As described in some working examples herein, methods of the present invention may be conducted using a short synthetic 16-base pair (bp) dsRNA, which identifies key features of the mode of protein RNA interaction. This dsRNA molecule has a sequence derived from a commonly used 29-base pair dsRNA-binding substrate which can be generated in small quantities by annealing the sense and antisense transcripts of the polylinker of the pGEM1 plasmid (Qian et al., 1995). Based on sedimentation equilibrium measurements, the stoichiometry of the binding of NS1A(1-73) to this synthetic 16-bp dsRNA duplex in solution is approximately 1:1 (one protein dimer with one dsRNA duplex molecule), with a bimolecular dissociation constant (K_{d}) in the micromolar range. The applicants propose this as a suitable dsRNA substrate molecule for use in high throughput binding assays. NMR chemical shift perturbation experiments demonstrate that the dsRNA-binding epitope of NS1A(1-73) is associated with antiparallel helices 2 and 2′, as has been previously indicated by site-directed mutagenesis studies (Wang et al., 1999). Circular dichroism (CD) spectra of the purified NS1A(1-73)-dsRNA complex are very similar to the sum of CD spectra of free dsRNA and NS1A(1-73), demonstrating that little or no change in the conformations of either the protein or its A-form dsRNA target occur as a result of binding. Moreover, because it is shown that NS1A(1-73) binds to neither the corresponding DNA-DNA duplex nor a DNA-RNA hybrids duplex, NS1A(1-73) appears to recognize specific conformational features of canonical A-form RNA, thus highlighting yet another way in which the methods of the present invention exquisitely mimics the interaction between the NS1 protein of influenza and its host.

Methods of the present invention are advantageously practiced in the context of a high throughput in vitro assay. In this embodiment of the invention, the assay system could use either or both of the standard methods of fluorescence resonance energy transfer or fluorescence polarization with labeled dsRNA molecules, either NS1A or NS1A(1-73), or NS1B or NS1B(1-93) molecules to monitor interactions between these protein targets and various dsRNA duplexes and to measure binding affinities. These assays would be used to screen compounds to identify molecules, which inhibit the interactions between the NS1 targets and the RNA substrates, based on the above-disclosed structure of the NS1 protein.

A wide variety of compounds may be tested for inhibitory activity against influenza virus in accordance with the present invention, including random and biased compound libraries. Biased compound libraries may be designed using the particular structural features of the NS1 target-RNA substrate interaction sites e.g., deduced on the basis of published results. See, e.g., Chien, et al., Nature Struct. Biol. 4:891-95 (1997); Liu, et al., Nature Struct. Biol. 4:896-899 (1997); and Wang, et al., RNA 5:195-205 (1999).

SCREENING ASSAYS FOR COMPOUNDS THAT INTERFERE WITH THE INTERACTION OF NS1A PROTEIN AND dsRNA REQUIRED FOR VIRAL REPLICATION: The NS1 protein of influenza virus, or a dsRNA binding domain thereof, and dsRNA which interact and bind are sometimes referred to herein as “binding partners”. Any of a number of assay systems may be utilized to test compounds for their ability to interfere with the interaction of the binding partners. However, rapid high throughput assays for screening large numbers of compounds, including but not limited to ligands (natural or synthetic), peptides, or small organic molecules, are preferred. Compounds that are so identified to interfere with the interaction of the binding partners should be further evaluated for antiviral activity in cell based assays, animal model systems and in patients as described herein. The basic principle of the assay systems used to identify compounds that interfere with the interaction between the NS1 protein of influenza virus, or a dsRNA binding domain thereof, and dsRNA involves preparing a reaction mixture containing the NS1 protein of influenza virus, or a dsRNA binding domain thereof, and dsRNA under conditions and for a time sufficient to allow the two binding partners to interact and bind, thus forming a complex. In order to test a compound for inhibitory activity, the reaction is conducted in the presence and absence of the test compound, i.e., the test compound may be initially included in the reaction mixture, or added at a time subsequent to the addition of NS1 protein of influenza virus, or a dsRNA binding domain thereof, and dsRNA; controls are incubated without the test compound or with a placebo. The formation of any complexes between the NS1 protein of influenza virus or a dsRNA binding domain thereof and the dsRNA is then detected. The formation of a complex in the control reaction, but not in the reaction mixture containing the test compound indicates that the compound interferes with the interaction of the NS1 protein of influenza virus or a dsRNA binding domain thereof and the dsRNA.

Still another aspect of the present invention comprises a method of virtual screening for a compound that can be used to treat influenza virus infections comprising using the structure of a NS1 protein or a dsRNA binding domain thereof NS1A(1-73) or NS1B(1-93), and the three dimensional coordinates of a model of the NS1-RNA complex in a drug screening assay.

Another aspect of the present invention comprises a method of using the three dimensional coordinates of the model of the complex for designing compound libraries for screening.

Accordingly, the present invention provides methods of identifying a compound or drug that can be used to treat influenza virus infections. One such embodiment comprises a method of identifying a compound for use as an inhibitor of the NS1 protein of influenza virus or a dsRNA binding domain thereof and a dataset comprising the three-dimensional coordinates obtained from the NS1 protein of influenza A or B virus or a dsRNA binding domain thereof. Preferably, the selection is performed in conjunction with computer modeling.

In one embodiment the potential compound is selected by performing rational drug design with the three-dimensional coordinates determined for the NS1 protein of influenza virus, or a dsRNA binding domain thereof. As noted above, preferably the selection is performed in conjunction with computer modeling. The potential compound is then contacted with and interferes with the binding of the NS1 protein of influenza virus or a dsRNA binding domain thereof and dsRNA, and the inhibition of binding is determined (e.g., measured). A potential compound is identified as a compound that inhibits binding of the NS1 protein of influenza virus or a dsRNA binding domain thereof and dsRNA when there is a decrease in binding. Alternatively, the potential compound is contacted with and/or added to influenza virus infected cell culture and the growth of the virus culture is determined. A potential compound is identified as a compound that inhibits viral growth when there is a decrease in the growth of the viral culture.

In a preferred embodiment, the method further comprises molecular replacement analysis and design of a second-generation candidate drug, which is selected by performing rational drug design with the three-dimensional coordinates determined for the drug. Preferably the selection is performed in conjunction with computer modeling. The candidate drug can then be tested in a large number of drug screening assays using standard biochemical methodology exemplified herein. In these embodiments of the invention the three-dimensional coordinates of the NS1A protein and the model of NS1A-dsRNA complex or the model of NS1B-dsRNA complex provide methods for (a) designing inhibitor library for screening, (b) rational optimization of lead compounds, and (c) virtual screening of potential inhibitors.

Other assay components and various formats in which the methods of the present invention may be practiced are described in the subsections below.

ASSAY COMPONENTS: One of the binding partners used in the assay system may be labeled, either directly or indirectly, to measure extent of binding between the NS1 protein or dsRNA binding portion, and the dsRNA. Depending upon the assay format as described in detail below, extent of binding may be measured in terms of complexation between NS1 protein of influenza virus, or a dsRNA binding domain thereof, and dsRNA, or extent of disassocation of a pre-formed complex, in the presence of the candidate compound. Any of a variety of suitable labeling systems may be used including but not limited to radioisotopes such as ¹²⁵I; enzyme labelling systems that generate a detectable colorimetric signal or light when exposed to substrate; and fluorescent labels.

Where recombinant DNA technology is used to produce the NS1 protein of influenza virus, or a dsRNA binding domain thereof, and dsRNA binding partners of the assay it may be advantageous to engineer fusion proteins that can facilitate labeling, immobilization and/or detection. For example, the coding sequence of the NS1 protein of influenza virus, or a dsRNA binding domain thereof, can be fused to that of a heterologous protein that has enzyme activity or serves as an enzyme substrate in order to facilitate labeling and detection. The fusion constructs should be designed so that the heterologous component of the fusion product does not interfere with binding of the NS1 protein of influenza virus, or a dsRNA binding domain thereof, and dsRNA.

Indirect labeling involves the use of a third protein, such as a labeled antibody, which specifically binds to NS1 protein of influenza virus, or a dsRNA binding domain thereof. Such antibodies include but are not limited to polyclonal, monoclonal, chimeric, single chain, Fab fragments and fragments produced by an Fab expression library.

For the production of antibodies, various host animals may be immunized by injection with the NS1 protein of influenza virus, or a dsRNA binding domain thereof. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies may be prepared by using any technique which provides for the production of antibody molecules by continuous cell lines in culture. These include but are not limited to the hybridoma technique originally described by Kohler and Milstein, (Nature, 1975, 256:495-497), the human B-cell hybridoma technique (Kosbor et al., 1983, Immunology Today, 4:72, Cote et al., 1983, Proc. Natl. Acad. Sci., 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce single chain antibodies specific to the NS1 protein of influenza virus or a dsRNA binding domain thereof.

Antibody fragments, which recognize specific epitopes may be generated by known techniques. For example, such fragments include but are not limited to: the F(ab′)2 fragments which can be produced by pepsin digestion of the antibody molecule and the Fab fragments which can be generated by reducing the disulfide bridges of the F(ab′)2 fragments. Alternatively, Fab expression libraries may be constructed (Huse et al., 1989, Science, 246:1275-1281) to allow rapid and easy identification of monoclonal Fab fragments with the desired specificity.

ASSAY FORMATS: The assay can be conducted in a heterogeneous or homogeneous format. Heterogeneous assays involve anchoring one of the binding partners onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the binding partners, e.g., by competition, can be identified by conducting the reaction in the presence of the test substance; i.e., by adding the test substance to the reaction mixture prior to or simultaneously with the NS1 protein of influenza virus, or a dsRNA binding domain thereof, and dsRNA. On the other hand, test compounds that disrupt preformed complexes, e.g. compounds with higher binding constants that displace one of the binding partners from the complex, can be tested, by adding the test compound to the reaction mixture after complexes have been formed. The various formats are described briefly below.

In a heterogeneous assay system, one binding partner, e.g., either the NS1 protein of influenza virus, or a dsRNA binding domain thereof, or dsRNA, is anchored onto a solid surface, and its binding partner, which is not anchored, is labeled, either directly or indirectly. In practice, microtiter plates are conveniently utilized. The anchored species may be immobilized by non-covalent or covalent attachments. Alternatively, an immobilized antibody specific for the NS1 protein of influenza virus, or a dsRNA binding domain thereof may be used to anchor the NS1 protein of influenza virus, or a dsRNA binding domain thereof to the solid surface. The surfaces may be prepared in advance and stored.

In order to conduct the assay, the binding partner of the immobilized species is added to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the binding partner was pre-labeled, the detection of label immobilized on the surface indicates that complexes were formed. Where the binding partner is not pre-labeled, an indirect label can be used to detect complexes anchored on the surface; e.g., using a labeled antibody specific for the binding partner (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for the NS1 protein of influenza virus or a dsRNA binding domain thereof to anchor any complexes formed in solution. Again, depending upon the order of addition of reactants to the liquid phase, test compounds which inhibit complex or which disrupt preformed complexes can be identified.

In other embodiments of the invention, a homogeneous assay can be used. In this approach, a preformed complex of the influenza viral NS1 protein or dsRNA binding domain thereof and dsRNA is prepared in which one of the binding partners is labeled, but the signal generated by the label is quenched due to complex formation (see, e.g., U.S. Pat. No. 4,109,496 by Rubenstein, which utilizes this approach for immunoassays). The addition of a test substance that competes with and displaces one of the binding partners from the preformed complex will result in the generation of a signal above background. In this way, test substances, which disrupt the NS1 protein of influenza virus, or a dsRNA binding domain thereof, and dsRNA interaction can be identified.

For example, in a particular embodiment the NS1 protein of influenza virus, or a dsRNA binding domain thereof, can be prepared for immobilization using recombinant DNA techniques described supra. Its coding region can be fused to the glutathione-S-transferase (GST) gene using the fusion vector pGEX-5X-1, in such a manner that its binding activity is maintained in the resulting fusion protein. NS1 protein or a dsRNA binding domain thereof can be purified and used to raise a monoclonal antibody, specific for NS1 or an NS1 fragment, using methods routinely practiced in the art and described above. This antibody can be labeled with the radioactive isotope ¹²⁵I, for example, by methods routinely practiced in the art. In a heterogeneous assay, e.g., the GST-NS1 fusion protein can be anchored to glutathione-agarose beads. dsRNA can then be added in the presence or absence of the test compound in a manner that allows dsRNA to interact with and bind to the NS1 portion of the fusion protein. After the test compound is added, unbound material can be washed away, and the NS1-specific labeled monoclonal antibody can be added to the system and allowed to bind to the complexed binding partners. The interaction between NS1 and dsRNA can be detected by measuring the amount of radioactivity that remains associated with the glutathione-agarose beads. A successful inhibition of the interaction by the test compound will result in a decrease in measured radioactivity.

Alternatively, the GST-NS1 fusion protein and dsRNA can be mixed together in liquid in the absence of the solid glutathione-agarose beads. The test compound can be added either during or after the binding partners are allowed to interact. This mixture can then be added to the glutathione-agarose beads and unbound material is washed away. Again the extent of inhibition of the binding partner interaction can be detected by measuring the radioactivity associated with the beads.

In accordance with the invention, a given compound found to inhibit one virus may be tested for general antiviral activity against a wide range of different influenza viruses. For example, and not by way of limitation, a compound which inhibits the interaction of influenza A virus NS1 with dsRNA by binding to the NS1 binding site can be tested, according to the assays described infra, against different strains of influenza A viruses as well as influenza B virus strains.

To select potential lead compounds for drug development, the identified inhibitors of the interaction between NS1 targets and RNA substrates may be further tested for their ability to inhibit replication of influenza virus, first in tissue culture and then in animal model experiments. The lowest concentrations of each inhibitor that effectively inhibits influenza virus replication will be determined using high and low multiplicities of infection.

VIRAL GROWTH ASSAYS: The ability of an inhibitor identified in the foregoing assay systems to prevent viral growth can be assayed by plaque formation or by other indices of viral growth, such as the TCID₅₀ or growth in the allantois of the chick embryo. In these assays, an appropriate cell line or embryonated eggs are infected with wild-type influenza virus, and the test compound is added to the tissue culture medium either at or after the time of infection. The effect of the test compound is scored by quantitation of viral particle formation as indicated by hemagglutinin (HA) titers measured in the supernatants of infected cells or in the allantoic fluids of infected embryonated eggs; by the presence of viral plaques; or, in cases where a plaque phenotype is not present, by an index such as the TCID₅₀ or growth in the allantois of the chick embryo, or with a hemagglutination assay. An inhibitor can be scored by the ability of a test compound to depress the HA titer or plaque formation, or to reduce the cytopathic effect in virus-infected cells or the allantois of the chick embryo, or by its ability to reduce viral particle formation as measured in a hemagglutination assay.

ANIMAL MODEL ASSAYS: The most effective inhibitors of virus replication identified by the processes of the present invention can then be used for subsequent animal experiments. The ability of an inhibitor to prevent replication of influenza virus can be assayed in animal models that are natural or adapted hosts for influenza. Such animals may include mammals such as pigs, ferrets, mice, monkeys, horses, and primates, or birds. As described in detail herein, such animal models can be used to determine the LD₅₀ and the ED₅₀ in animal subjects, and such data can be used to derive the therapeutic index for the inhibitor of the NS1A(1-73) or NS1B(1-93) and dsRNA interaction.

Optimization of design of lead compounds may also be aided by characterizing binding sites on the surface of the NS1 protein or dsRNA binding domain thereof by inhibitors identified by high throughput screening. Such characterization may be conducted using chemical shift perturbation NMR together with NMR resonance assignments. NMR can determine the binding sites of small molecule inhibitors for RNA. Determining the location of these binding sites will provide data for linking together multiple initial inhibitor leads and for optimizing lead design.

PHARMACEUTICAL PREPARATIONS AND METHODS OF ADMINISTRATION: The identified compounds that inhibit viral replication can be administered to a patient at therapeutically effective doses to treat viral infection. A therapeutically effective dose refers to that amount of the compound sufficient to result in amelioration of symptoms of viral infection.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds, which exhibit large therapeutic indices are preferred. While compounds that exhibit toxic side effects may be used, care should be taken to design a delivery system that targets such compounds to the site of infection in order to minimize damage to uninfected cells and reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal infection, or a half-maximal inhibition) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

The invention is not limited to the embodiments described herein and may be modified or varied without departing from the scope of the invention

### Example 1

PROTEIN SAMPLE PREPARATION: E. coli BL21(DE3) cell cultures were transformed with a pET11a expression vector encoding NS1A(1-73), grown at 37° C., and then induced with 1 mM IPTG at OD₆₀₀=0.6 for 5 hours in MJ minimal medium (Jansson et al., (1996) J. Biomol. NMR 7, 131-141.) containing uniformly enriched ¹⁵NH₄Cl and ¹³C₆-glucose as the sole nitrogen and carbon sources, respectively. Cells were broken by sonication, followed by centrifugation at 100,000×g at 4° C. for 1 hour. Proteins were then purified from the supernatant by ion exchange and gel filtration chromatography using Pharmacia FPLC systems according to a procedure described elsewhere. (Qian et al., (1995) RNA 1, 948-956.) The overall yield of purified NS1A(1-73) was about 5 mg/l of culture medium. Protein concentrations were determined by absorbance at 280 nm (A₂₈₀) using a molar extinction coefficient (ε₂₈₀) for the monomer of 5750 M⁻¹ cm⁻¹.

### Example 2

SYNTHESIS AND PURIFICATION OF RNA OLIGOMERS: Two single-stranded (ss) 16-nucleotide (16-nt) RNAs, CCAUCCUCUACAGGCG (sense) and CGCCUGUAGAGGAUGG (antisense), were chemically synthesized using standard phosphoramidite chemistry (Wincott et al., (1995) Nucleic Acids Res. 23, 2677-2684) on a DNA/RNA synthesizer Model 392 (Applied Biosystems, Inc.) Both RNA oligomers were then desalted over Bio-Rad Econo-Pac 10DG columns and purified by preparative gel electrophoresis on 20% (w/v) acrylamide, 7M urea denaturing gels. The appropriate product bands, visualized by UV shadowing, were cut out, crushed, and extracted into 90 mM Tris-borate, 2 mM EDTA, pH 8.0 buffer by gentle rocking overnight. The resulting solutions were concentrated by lyophilization and desalted again using Econo-Pac 10DG columns. Purified RNA oligomers are then lyophilized and stored at −20°. Analogous 16-nt sense and antisense DNA strands containing the same sequence can be purchased from Genosys Biotechnologies, Inc. Concentrations of nucleic acid samples were calculated on the basis of absorbance at 260 nm (A₂₆₀) using the following molar extinction coefficients (₂₆₀, M⁻¹ cm⁻¹ at 20° C.): (+) RNA, 151 530; (−) RNA, 165 530; (+) DNA, 147 300; (−) DNA, 161 440; dsRNA, 262 580; RNA/DNA, 260 060; DNA/RNA, 273 330; dsDNA, 275 080. The extinction coefficients for the single strands were calculated from the extinction coefficients of monomers and dimers at 20° C. (Cantor et al., (1965) J. Mol. Biol. 13, 65-77) assuming that the molar absorptivity is a nearest-neighbor property and that the oligonucleotides are single-stranded at 20° C. (Hung et al., (1994), Nucleic Acids Res. 22, 4326-4334). Molar extinction coefficients for the duplexes were calculated from the A₂₆₀ values at 20 and 90° C. using the following expression: ε_{(260, 20°)}[A_{.260, 20°)}/A_{.260, 90°)}]×ε_{.260, 90°, calc)}, where ε_{.260, 90°, calc)} is the molar extinction coefficient at 90° C. obtained from the sum of the single strands assuming complete dissociation of the duplex at this temperature.

### Example 3

POLYACRYLAMIDE GEL SHIFT BINDING ASSAY: The single-stranded 16-nt synthetic RNA and DNA oligonucleotides were labeled at their 5′ ends with [γ³²P]ATP using T4 polynucleotide kinase and purified by denaturing urea-PAGE. Approximate 1:1 molar ratios of single-stranded (ss) sense RNA (or DNA) and antisense RNA (or DNA) were mixed in 50 mM Tris, 100 mM NaCl, pH 8.0 buffer. Solutions were heated to 90° C. for two minutes and then slowly cooled down to room temperature to anneal the duplexes. NS1A(1-73), final concentration of 0.4 μM, was added to each of the four double-stranded (ds) nucleic acids (dsRNA (RR), RNA-DNA (RD) and DNA-RNA (DR) hybrids, and dsDNA (DD), 10,000 cpm, final concentration ≈1 nM) in 20 μl of binding buffer (50 mM Tris-glycine, 8% glycerol, 1 mM dithiothreitol, 50 ng/μl tRNA, 40 units of RNasin, pH 8.8). The reaction mixture was incubated on ice for 30 min. The protein-nucleic acid complexes were resolved from free ds or ss oligomers by 15% nondenaturing PAGE at 150 V for 6 hours in 50 mM Tris-borate, 1 mM EDTA, pH 8.0 at 4° C. The gel was then dried and analyzed by autoradiography.

### Example 4

ANALYTICAL GEL FILTRATION CHROMATOGRAPHY: Micromolar solutions of the four 16-nt duplexes (RR, RD, DR, and DD) were prepared 10 mM potassium phosphate, 100 mM KCl, 50 μM EDTA, pH 7.0 buffer and annealed as described above. These duplexes are then purified from unannealed or excess ss species using a Superdex-75 HR 10/30 gel filtration column (Pharmacia), and adjusted to a duplex concentration of 4 μM. Each ds nucleic acid was then combined with 1.5 mM NS1A(1-73) (monomer concentration) to give a 1:1 molar ratio of protein to duplex. Gel filtration chromatography can be performed on a Superdex 75 HR 10/30 column (Pharmacia). This column is calibrated using four standard proteins: albumin (67 kDa), ovalbumin (43 kDa), chymotrypsinogen A (25 kDa), and ribonuclease A (13.7 kDa). Chromatography is carried out in 10 mM potassium phosphate and 100 mM KCl, 50 μM EDTA, pH 7.0 at 20° C. using a flow rate of 0.5 ml/min. Samples of protein-duplex in a 1:1 molar ratio are applied to the column, and the fractions are monitored for the presence of nucleic acid by their A₂₆₀; the contribution to the UV absorbance from NS1A(1-73) can be ignored due to its relatively small ε₂₆₀ compared to the nucleic acid duplexes.

### Example 5

PURIFICATION OF THE NS1A(1-73)-DSRNA COMPLEX: The fraction corresponding to the first peak shown in the gel filtration chromatography of 1:1 molar ratio NS1A(1-73) dimer and dsRNA mixture was collected and concentrated to less than 1 ml using Centricon concentrators (Amicon, Inc.). This concentrated sample was then reloaded onto the same gel filtration column and the main fraction is collected again. The concentration of this purified NS1A(1-73)-dsRNA complex was determined by measuring the UV absorbance at 260 nm. The purity and stability of this complex was also examined using analytical gel filtration by loading 100 μl samples at 4 μM immediately following preparation and after 1 month.

### Example 6

SEDIMENTATION EQUILIBRIUM: Sedimentation equilibrium experiments were carried out using a Beckman XL-I instrument at 25° C. Short column runs using Beckman eight-channel 12 mm path charcoal-Epon cells at speeds 30K to 48K rpm were conducted for NS1A(1-73) and dsRNA loading concentrations of 0.2-2 mg/ml and 0.2-0.6 mg/ml, respectively, in order to independently evaluate the behavior of these free components. Data were acquired using a Rayleigh interference optical system. To investigate the association behavior of the NS1A(1-73) dimer and dsRNA, long column runs were conducted using Beckman six-channel (1.2 cm path) charcoal-Epon cells at speeds of 16K to 38K rpm using samples of the complex purified by gel filtration chromatography. These data were acquired using a UV absorbance optical system at 260 nm and loading concentrations of 0.3, 0.5 and 0.6 absorbance units. To ensure sample equilibration, measurements were taken every 0.5 h for 4 h for the short column and every 1 to 6 h for 8 to 28 h for the long column. Equilibrium was determined to have been established when the difference between two scans taken 1 hour apart, calculated using program WINMACH (developed by Yphantis, D. A. and Larry, J, Distributed by the National Analytical Ultracentrifugation Facility at The University of Connecticut) was within 0.005-0.008 fringes for the Rayleigh interference optics, or about 0.005 OD for absorbance optics.

Data analysis was performed using program WINNL106, a Windows 95 version based on the original nonlinear least-squares programs NONLIN (Johnson et al., 1981). The data were either fit separately for each data set at a specific loading concentration and speed, or jointly by combining several sets of data with different loading concentrations and/or speeds. The global fit refers to the fitting conducted by using all data sets and with the association constant lnK treated as a common parameter. To avoid the complications caused by the deviation from Beer's law, the absorbance data were edited with a cutoff value of OD≦1.0 from the base region, unless otherwise noted.

The partial specific volume of NS1A(1-73), v_{NS1}, and the solvent density, ρ, are calculated to be 0.7356 and 1.01156, respectively, at 25° C. using the program Sednterp (Laue et al., 1992). The specific volume of dsRNA, v_{RNA}, is determined experimentally to be 0.5716 by sedimentation equilibrium of dsRNA samples (see Results for details). The specific volume of the NS1A(1-73)-dsRNA complex, v_{complex}, is calculated to be 0.672 assuming a 1:1 stoichiometry, using the method of Cohn and Edsall (Cohn & Edsall, 1943).

### Example 7

CALCULATION OF THE DISSOCIATION CONSTANT: The calculation of the dissociation constant of a 1:1 NS1A(1-73)-dsRNA complex was based upon the assumption that there are equal molar amounts of free NS1A(1-73) protein and free dsRNA in the original solution. This assumption is valid if the gel-filtration purified samples of the complex used in these measurements is in fact a 1:1 stoichiometry. In this case, the amount of free dsRNA and free NS1A(1-73) correspond to that which has dissociated from the 1:1 complex. In addition, since the reduced molecular weight (defined below in Eq. 2) of NS1A(1-73) dimer and dsRNA differ only by 3%, the two free macromolecules are treated as the same hydrodynamic species during sedimentation. The concentration distribution of the ith species of an ideal system at sedimentation equilibrium can be expressed as

Cᵢ(r)=Cᵢ(r′)e^{σ}^{ᵢ}^{(r}^{²}^{/2-r′}^{²}^{/2)} (Eq. 1)

(Johnson et al. 1981) where C(r)ᵢ is the weight concentration of the ith component at a radius r, r′ is a reference position inside the solution column. The σᵢ in above equation is the reduced molecular weight (Yphantis & Waugh, 1956):

σᵢ=Mᵢ(1− v̅ᵢρ)ω²/RT. (Eq. 2)

The Mᵢ and v̅ᵢ in Eq. 2 are the molecular weight and the partial specific volume of the ith species, R is the gas constant, T is the absolute temperature and ω is the angular velocity. The concentration is normally expressed in weight concentration scale (mg/ml), however, for our case it is more convenient to use the molar concentration m, with mᵢ=Cᵢ/Mᵢ.

Based on the principle of conservation of mass (Van Holde &

$${m}_{RNA , t}^{0} ( {r}_{b}^{2} / 2 - {r}_{m}^{2} / 2 ) = {\int }_{{r}_{b}}^{{r}_{m}} {m ( r )}_{RNA , free} {e}^{{σ}_{RNA} ( {r}^{2} / 2 - {r}^{′2} / 2 )} r d r + {\int }_{{r}_{b}}^{{r}_{m}} {m ( r )}_{x} {e}^{{σ}_{x} ( {r}^{2} / 2 - {r}^{′2} / 2 )} r d r \qquad ( Eq . 3 )$$

Baldwin, 1958), the dsRNA can be expressed by

The quantity m⁰ refers to the concentration of the original solution, while m(r) refers to the concentration at radius r at sedimentation equilibrium. The subscripts “RNA,t”, “RNA,free” and “RNA,x” refer to the total amount of dsRNA, the free dsRNA and dsRNA in the NS1A(1-73)-dsRNA complex, respectively; rₘ and r_{b} are radius values at the meniscus and base of the solution column, respectively. In order to simplify the results to follow, r′ is set to be at the position of rₘ. Integration of equation 3 then yields:

$${m}_{RNA , t }^{0} ( {r}_{b}^{2} / 2 - {r}_{m}^{2} / 2 ) = \frac{\begin{matrix}{m ( {r}_{b} )}_{RNA , free} - \\ {m ( {r}^{′} )}_{RNA , free}\end{matrix}}{{σ}_{RNA}} + \frac{{m ( {r}_{b} )}_{x} - {m ( {r}^{′} )}_{x}}{{σ}_{x}} \qquad ( Eq . 4 )$$

where m(r_{b})_{RNA,free} and m(r_{b})_{RNA,x} are the concentrations of the dsRNA free and in complex with NS1A(1-73), respectively, at the base of the solution column. The same equation can also be expressed for NS1A(1-73) protein. Under the condition that m⁰_{RNA} equals m⁰_{NS1} the equation yields:

$$\frac{{m ( {r}^{′} )}_{RNA , free}}{{σ}_{RNA}} ( \frac{{m ( {r}_{b} )}_{RNA , free}}{{m ( {r}^{′} )}_{RNA , free}} - 1 ) = \frac{{m ( {r}_{b} )}_{NS 1 , free}}{{σ}_{NS 1}} ( \frac{{m ( {r}_{b} )}_{NS 1 , free}}{{m ( {r}^{′} )}_{NS 1 , free}} - 1 ) . \qquad ( Eq . 5 )$$

Making use of the fact that σ_{RNA} [symbol] σ_{NS1}, for this particular protein:RNA complex, Eq. 5 demonstrates that m(r′)_{RNA,free}=m(r′)_{NS1,free} at the reference position, and thus, m(r)_{RNA,free}=m(r)_{NS1,free} at any radius r.

Finally, the absorbance at radius r at sedimentation equilibrium is expressed as:

A₂₆₀(r)=Eₓm(r′)_{RNA}e^{σ}^{_{RNA}}^{(r}^{²}^{/2-r′}^{²}^{/2)}+(1/Eₓ)Kₐ[Eₓm(r′)_{RNA}e^{σ}^{_{RNA}}^{(r}^{²}^{/2-r′}^{²}^{/2)}]² (Eq. 6)

In above equation, Eₓ=(ε_{RNA}+ε_{NS1})1, where ε is the extinction coefficient and 1 is the optical path length. The Kₐ is the association constant in molar concentration scale, and is expressed as a function of mₓ and m_{RNA} (Eq. 7), under the condition m_{RNA}=m_{NS1}.

Ka=mx/mRNA2 (Eq. 7)

Thus, the association system of NS1A(1-73) and dsRNA is reduced to a simple system of two components during sedimentation. It can be easily fit with an ideal monomer-dimer self-associating model of NONLIN with the fit parameter K₂=Kₐ/Eₓ, and the dissociation constant of the NS1A(1-73)-dsRNA complex, K_{d}, is calculated from the following equation:

K_{D}=1/(EₓK₂). (Eq. 8).

### Example 8

NMR SPECTROSCOPY: All NMR data were collected at 20° C. on Varian INOVA 500 and 600 NMR spectrometer systems equipped with four channels. The programs VNMR (Varian Associates), NMRCompass (Molecular Simulations, Inc.), and AUTOASSIGN (Zimmerman et al., (1997) J. Mol. Biol. 269, 592-610) were used for data processing and analysis. Proton chemical shifts were referenced to internal 2,2-dimethyl-2-silapentane-5-sulfonic acid; ¹³C and ¹⁵N chemical shifts were referenced indirectly using the respective gyromagnetic ratios, ¹³C:¹H (0.251449530) and ¹⁵N:¹H (0.101329118). (Wishart et al., (1995) J. Biomol. NMR 6, 135-140.)

### Example 9

SEQUENCE SPECIFIC ASSIGNMENTS OF NS1A(1-73): NMR samples of free ¹³C, ¹⁵N—NS1A(1-73) used for assignment were prepared at a dimer protein concentration of 1.0 to 1.25 mM in 270 μl of 95% H₂O/5% D₂O solutions containing 50 mM ammonium acetate and 1 mM NaN₃ at pH 6.0 in Shigemi susceptibility-matched NMR tubes. Backbone ¹H, ¹³C, ¹⁵N, and ¹³C resonance assignments were determined by automated analysis of triple-resonance NMR spectra of ¹³C, ¹⁵N-enriched proteins using the computer program AUTOASSIGN (Zimmerman et al., (1997) J. Mol. Biol. 269, 592-610). The input for AUTOASSIGN includes peak lists from 2D ¹H-15N HSQC and 3D HNCO spectra along with peak lists from three intraresidue [HNCA, CBCANH, and HA(CA)NH] and three interresidue [CA(CO)NH, CBCA(CO)NH, and HA(CA)(CO)NH] experiments. Details of these pulse sequences and optimization parameters were reviewed elsewhere (Montelione et al., (1999), Berliner, L. J., and Krishna, N. R., Eds, Vol. 17, pp 81-130, Kluwer Academic/Plenum Publishers, New York). Peak lists for AUTOASSIGN were generated by automated peak-picking using NMRCompass and then manually edited to remove obvious noise peaks and spectral artifacts. Side chain resonance assignments (except for the ¹³C assignments of aromatic side chains) were then obtained by manual analysis of 3D HCC(CO)NH TOCSY (Montelione et al., (1992) J. Am. Chem. Soc. 114, 10974-10975), HCCH-COSY (Ikura et al., (1991) J. Biomol. NMR 1, 299-304) and ¹⁵N-edited TOCSY (Fesik et al., (1988) J. Magn. Reson. 78, 588-593) experiments and 2D TOCSY spectra recorded with mixing times of 32, 53, and 75 ms (Celda and Montelione (1993) J. Magn. Reson. Ser. B 101, 189-193).

### Example 10

NMR CHEMICAL SHIFT PERTURBATION EXPERIMENTS: ¹⁵N-enriched NS1A(1-73) was purified and prepared as described above. A 250 μl solution of ¹⁵N-enriched NS1A(1-73), 0.1 mM dimer, in 50 mM ammonium acetate, 1 mM NaN₃, 5% D₂O, pH 6.0 was first used for collecting the ¹H^{N}-¹⁵N HSQC spectrum of free protein. The 16-nt sense and antisense RNA strands in a 1:1 molar ratio were annealed in 200 mM ammonium acetate, pH 7.0, lyophilized three times, and dissolved in the same NMR sample buffer, for a final RNA duplex concentration of 10 mM. This highly concentrated dsRNA solution was then used to titrate the NMR sample of free ¹⁵N-enriched NS1A(1-73), making protein-dsRNA samples with the ratios of [dimeric protein] to [dsRNA] as 2:1, 1:1, 1:1.5, and 1:2. In order to prevent the precipitation of NS1A(1-73), these samples were prepared by slowly adding the free protein solution to the concentrated dsRNA. The HSQC spectra of free ¹⁵N-enriched NS1A(1-73) were acquired with 80 scans per increment and 200×2048 complex data points, and transformed into 1024×2048 points after zero-filling in the t1 dimension. HSQC spectra for the dsRNA titration experiments were collected with the same digital resolution using 320 scans per increment.

### Example 11

CD MEASUREMENTS: CD spectra were recorded in the 200-350 nm region at 20° C. using an Aviv Model 62-DS spectropolarimeter equipped with a 1 cm path-length cell. CD spectra for the four nucleic acid duplexes (RR, RD, DR, DD) were obtained on 1.1 ml, 4 μM samples in the phosphate buffer described above. Each duplex is then combined with 1.5 mM NS1A(1-73) (monomer concentration) to form a 1:1 molar ratio of protein to duplex. The CD spectra of these protein-duplex mixtures were collected under the same conditions, assuming that the total duplex concentration remained 4 μM for each sample. The CD spectra of a 1.1 ml samples of free NS1A(1-73) and column purified NS1A(1-73)-dsRNA complex, both at 4 μM in the same phosphate buffer, were also acquired. The calculated CD spectra of protein-duplex mixtures were obtained using the sum of CD data from free NS1A(1-73) and from each double-stranded nucleic acid alone. CD spectra were reported as ε_{L}-ε_{R}, in units of M⁻¹cm⁻¹ per mol nucleotide.

### Example 12

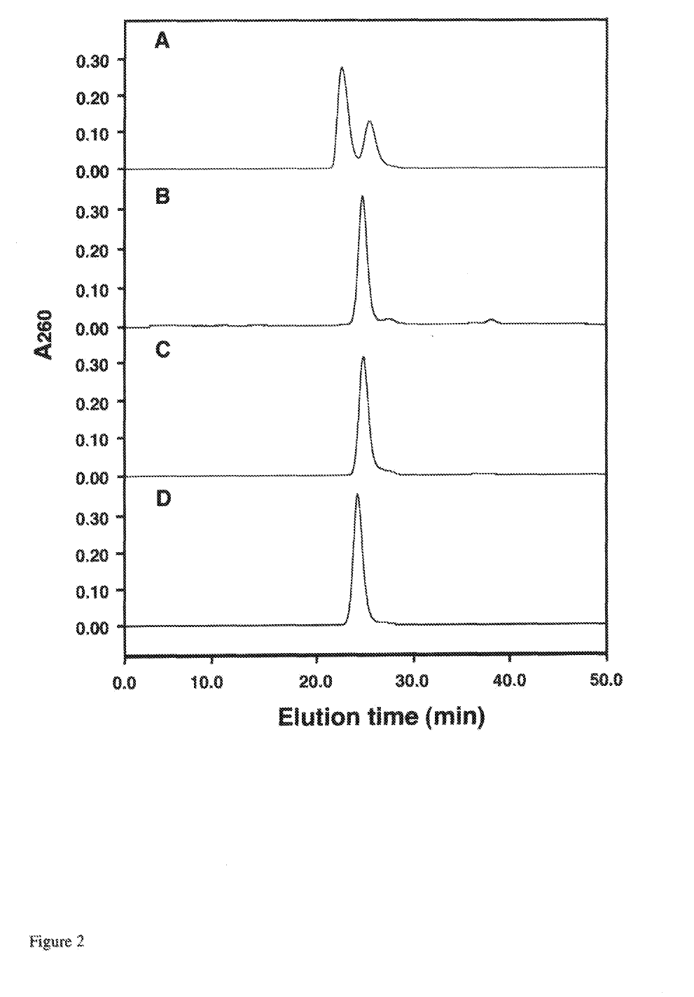

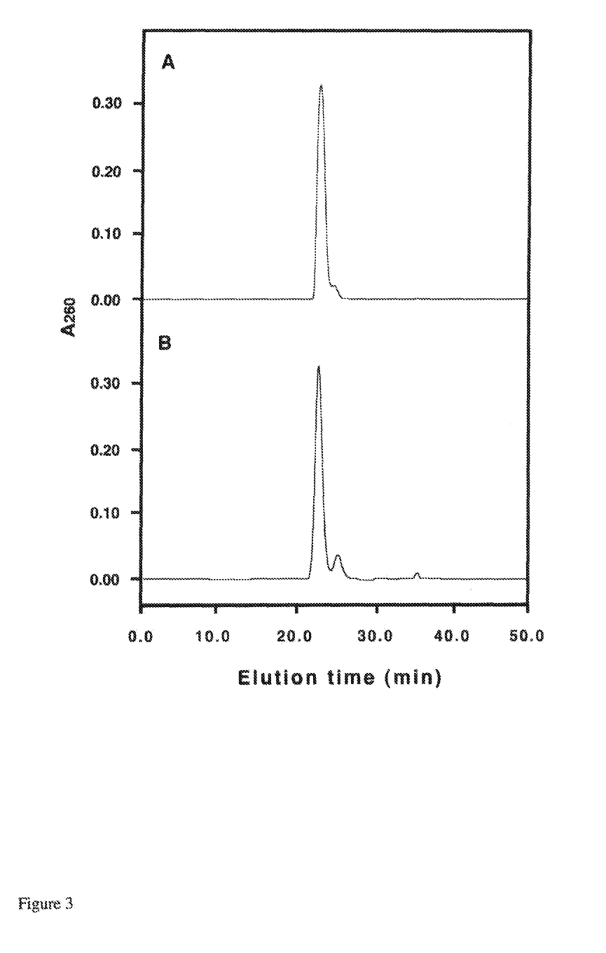

CHARACTERIZATION AND PURIFICATION OF NS1A(1-73)-DSRNA COMPLEX BY GEL FILTRATION CHROMATOGRAPHY: The four NS1A(1-73)-nucleic acid duplex mixtures described above were further analyzed for complex formation using analytical gel filtration chromatography. The NS1A(1-73)-dsRNA mixture showed two major peaks in the chromatographic profile monitored at 260 nm (FIG. 2A), whereas the mixtures containing dsDNA and RNA/DNA eluted as a single peak (FIGS. 2B, C, D). Since the chromatographic eluates were detected by absorbance at 260 nm, these chromatograms reflect the state(s) of the nucleic acid in these samples. In the dsRNA case (FIG. 2A), the faster and slower eluting peaks corresponded to the NS1A(1-73)-dsRNA complex and the unbound dsRNA duplex, respectively. The elution time and corresponding molecular weight (˜26 kDa) for the more rapidly eluting peak were consistent with a complex with a 1:1 stoichiometry (protein dimer to dsRNA). About 70% of the RNA and protein werte in the complex fraction under the chromatographic conditions used. No peak(s) corresponding to complex formation was observed for the other samples. These results provide further evidence that NS1A(1-73) binds exclusively to dsRNA, and not to dsDNA or the RNA/DNA hybrids studied here. Gel filtration chromatography was also used preparatively to purify NS1A(1-73)-dsRNA complex prior to subsequent experimentations (i.e., sedimentation equilibrium and CD) and to evaluate the long term stability of the complex (FIG. 3). Rechromatographic analysis of the freshly purified NS1A(1-73)-dsRNA complex yielded a single peak consistent with a relatively stable and pure complex (FIG. 3A). However, an increase in free dsRNA was observed after one month of storage at 4° C. (FIG. 3B), suggesting that the complex slowly and irreversibly dissociates over long periods of time.

### Example 13

SEDIMENTATION EQUILIBRIUM: FREE NS1A(1-73) AND DSRNA: Sedimentation equilibrium techniques are used to determine the stoichiometry and dissociation constant of complex formation between NS1A(1-73) and the 16-bp dsRNA duplex. First, short-column equilibrium runs are conducted on purified NS1A(1-73) protein and purified dsRNA samples with multiple loading concentrations and multiple speeds. The NS1A(1-73) protein exists as a dimer in solution with molecular weight of 16,851 g/mol, and no obvious signs of dissociation (data not shown). In some instances the NS1A(1-73) samples used for these sedimentation experiments include the presence of large nonspecific aggregates. The total amount of aggregate formation may vary with each sample and is separated from the dimer species at high speeds. This is indicative of a slow sample-dependent aggregation process. Consequently, samples of protein in complex with dsRNA are purified by gel filtration immediately prior to conducting sedimentation equilibrium measurements (see FIG. 3). The purified dsRNA sample behave as an ideal solution with a single component during sedimentation. The estimated reduced molecular weight obtained by fitting the data to the single component model of NONLIN does not change with the loading concentration and/or speed. This enables the calculation of the specific volume of dsRNA based on the estimated reduced molecular weight using Eqn. 2 (see above). The value obtained, v_{RNA}=0.57 units, agrees well with the typical partial specific volume values of DNA (0.55-0.59 units) and RNA (0.47-0.55 units) (Ralston, 1993). The fact that this value of v_{RNA} is closer to that of dsDNA than typical RNA samples, may be attributed to its double-stranded conformation. A conservative estimate of about 7% error in the reduced molecular weight translates into approximately the same error in the specific volume. In this analysis, it is assumed that the formation of the complex has no significant effect on the specific volume of the dsRNA and the NS1A(1-73) protein.

### Example 14

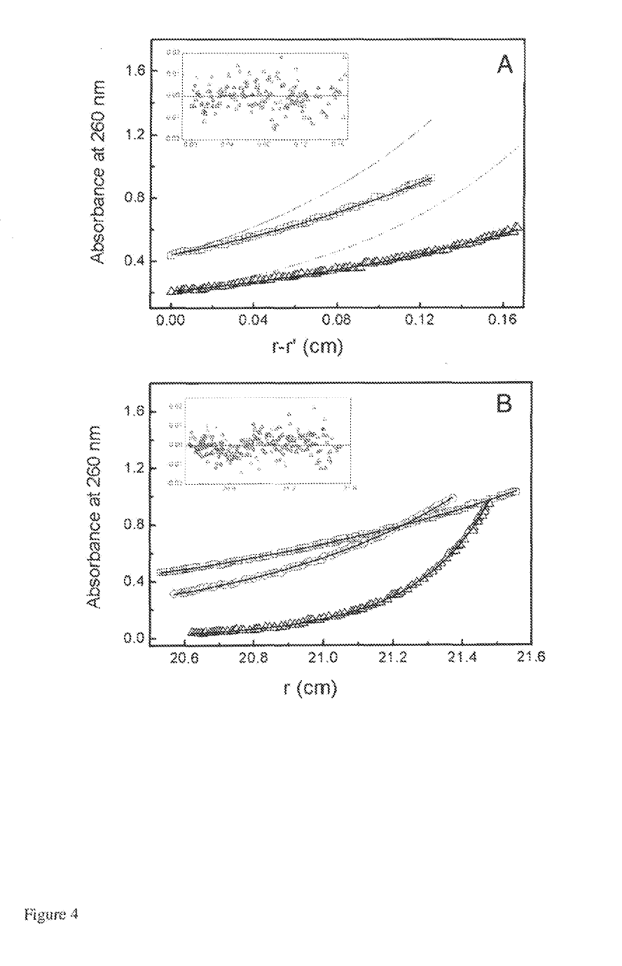

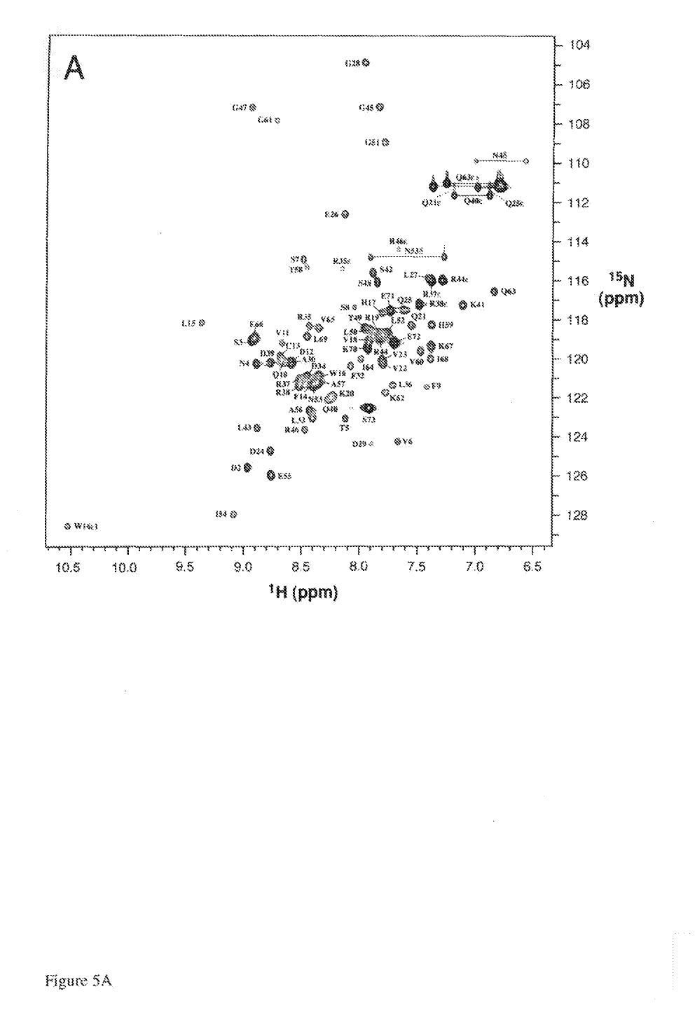

STOICHIOMETRY AND THERMODYNAMICS OF COMPLEX FORMATION BASED ON SEDIMENTATION EQUILIBRIUM: The association of NS1A(1-73) protein with dsRNA was studied using samples of purified NS1A(1-73)-dsRNA complex prepared as described above and validated as homogeneous by analytical gel filtration (FIG. 3A). The stoichiometry of the complex was determined on the basis of data collected at 16000 rpm (FIG. 4A). At this low speed the free dsRNA and NS1A(1-73) protein have a σᵢ value less than 0.5 (Eqn. 2). Under these slow speed conditions, the two lower molecular weight species (i.e., free NS1A(1-73) and free dsRNA) did not significantly redistribute and thus had baseline contributions to the absorbance profile. Accordingly, these data were fit to an ideal single component model using NONLIN (FIG. 4A and Table 3). The estimated apparent molecular weights (Mₐₚₚ) of ≈24.4 kDa were very close to that of a 1:1 NS1A(1-73)-dsRNA complex calculated from the corresponding amino acid and nucleic acid sequences. The relatively low RMS values and random residual plots (insert of FIG. 4A) indicated a good fit to a 1:1 stoichiometry. When the data were edited with an OD₂₆₀ cutoff value of 0.8 from the base of the solution column, the quality of the fit is further improved (Table 3). The estimated average molecular weight of 26,100 g/mole, was within ≈3% of the formula molecular weight of a 1:1 NS1A(1-73)-dsRNA complex. This shows that this purified NS1A(1-73)-dsRNA complex has a 1:1 stoichiometry. Based on the 1:1 stoichiometry, the data at three different loading concentrations and at three speeds were then fit to the equilibrium monomer-dimer model of NONLIN, in order to estimate the dissociation constant, K_{d} (FIG. 4B). Using this model, excellent fits to the data were obtained, as judged by the small RMS values and random residual plots. In order to verify that the fitting model is correct, the individual data sets were also fit separately or jointly using different combinations such as data of a single loading concentration at three different speeds, or data of different loading concentrations but at one speed, and so on. For each fit, several different models were compared. In all cases the monomer-dimer model emerged as the best. One exception was the data obtained at 16K rpm, which fit equally well to both the single component system and monomer-dimer models. It is also possible to edit the data with different cutoff values at the base of the cell; this leads to the final fitting results being relatively independent of the cutoff between 0.8 to 1.5 absorbance units. The K_{d} values calculated using Eq. 8 fall within a relatively narrow range, K_{d}=0.4-1.4 μM, depending on the specific fitting conducted.

TABLE 3
Apparent Molecular Weight of the
NS1A(1-73)-dsRNA Complex.

| | NONLIN fitting | | | | | |
|---|---|---|---|---|---|---|
| | O.D. cut off^{b} ~1.0 | | | O.D. cutoff ~0.8 | | |
| Cₜ^{0a} | RMS^{c} | Mₐₚₚ^{d} | Mₐₚₚ/Mₓ^{e} | RMS^{c} | Mₐₚₚ^{d} | Mₐₚₚ/Mₓ^{e} |
| 0.6 | 0.0061 | 27.5 | 1.02 | 0.0051 | 28.8 | 1.07 |
| 0.5 | 0.0043 | 23.3 | 0.86 | 0.0040 | 26.0 | 0.96 |
| 0.3 | 0.0063 | 24.9 | 0.92 | 0.0065 | 24.4 | 0.90 |
| Joint | 0.0056 | 24.4 | 0.91 | 0.0054 | 25.2 | 0.94 |
| fit | | | | | | |

^{a}The concentration of the initial solution measured by absorbance at 260 nm.
^{b}OD_{260 nm} data greater than the cutoff value were not included in the fit.
^{c}The root-mean-square value of fitting in units of absorbance.
^{d}The apparent molecular weight, in kg/mole, estimated by fitting the data of to an ideal solution with single component (FIG. 4A). The data were either fit individually at each loading concentration or jointly all three data sets together.
^{e}Ratio of apparent molecular weight (Mₐₚₚ) based on sedimentation equilibrium data to the molecular weight of a 1:1 NS1A(1-73): dsRNA complex calculated from the amino acid and nucleic acid sequence (Mₓ).

### Example 15

¹H, ¹⁵N, AND ¹³C RESONANCE ASSIGNMENTS FOR FREE NS1A(1-73): Essentially complete NMR resonance assignments for the free NS1A(1-73) protein, required for the analysis of its complex with dsRNA by NMR, were determined. In all, a total of 65/71 (92%) assignable ¹⁵N-¹H^{N} sites were assigned automatically using AUTOASSIGN (Zimmerman et al. (1997) J. Mol. Biol. 269, 592-610). This automated analysis provided 71/78H^{α}, 68/73 C^{α}, 64/71 C′, and 44/68 C^{β} resonance assignments via intraresidue and/or sequential connectivities. Subsequent manual analysis of the same triple-resonance data confirmed these results of AUTOASSIGN and also completed the resonance assignments for the remaining backbone atoms and 60/68 C^{β} atoms. All backbone resonances were assigned except Met¹ NH₂, Pro³¹N, and C′ of the C-terminal residue Ser⁷³ and Pro-preceding residue Ala³⁰. Complete side chain assignments of non-exchangeable protons and protonated carbons (the aromatic carbons are not included) were then obtained for all residues. With regard to exchangeable side chain groups, all Arg N^{ε}H, Gln N^{ε2}H, Asp N^{δ2}H, and Trp N^{ε1}H resonances were also assigned, but no Arg N^{η}H or hydroxyl protons of Ser and Thr were observed in these spectra. These ¹H, ¹³C, ¹⁵N chemical shift data for NS1A(1-73) at pH 6.0 and 20° C. have been deposited in BioMagResBank (http://www.bmrb.wisc.edu; accession number 4317).

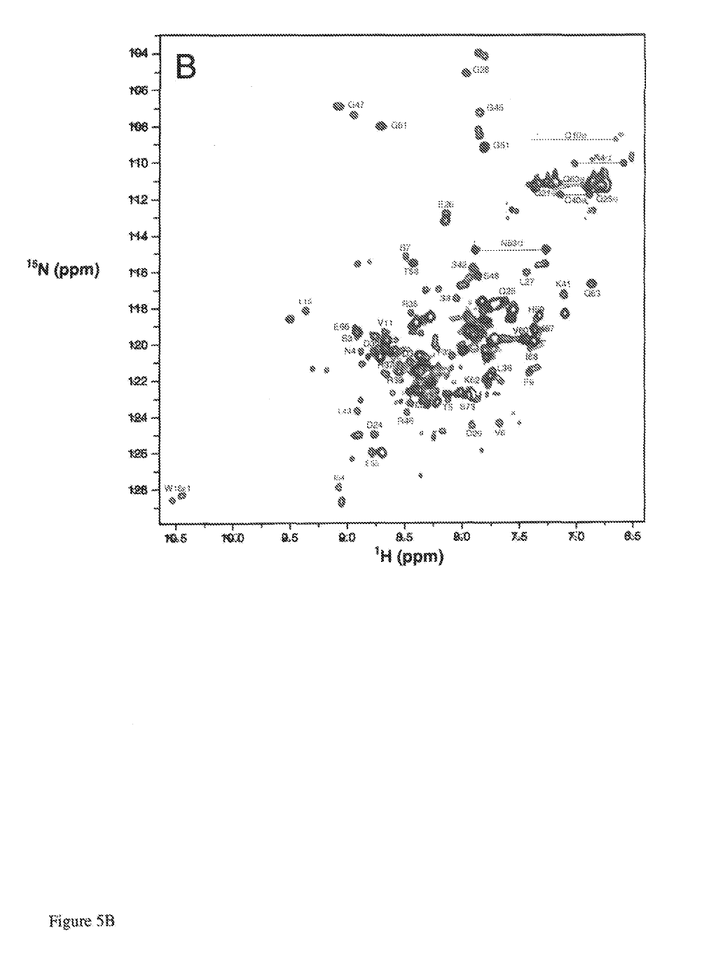

The ¹H-¹⁵N HSQC spectrum for ¹⁵N-enriched NS1A(1-73) at pH 6.0 and 20° C. is shown in FIG. 5. All backbone amide peaks (except for Pro³¹ and the N-terminal Met¹) were labeled, as are the side-chain resonances of Arg N^{ε}H, Gln N^{ε2}H, Asp N^{δ2}H, and Trp N^{ε1}H. Overall, the spectrum displayed reasonably good chemical shift dispersion, although there were a few degenerate ¹⁵N-¹H^{N} cross peaks. For example, residues Arg³⁷ and Arg³⁸ had almost the same chemical shifts for H^{N}, N, C′, C^{α}, H^{α}, and C^{β} resonances.

### Example 16

EPITOPE MAPPING BY CHEMICAL SHIFT PERTURBATION: Monitoring of the titration of ¹⁵N-enriched NS1A(1-73) was accomplished with the 16 by dsRNA by collecting a series of ¹H^{N}-¹⁵N HSQC spectra. The chemical shifts of both ¹H and ¹⁵N nuclei were sensitive to their local electronic environment and therefore are used as probes for interactions between the labeled protein and unlabeled RNA. The strongest perturbation of the electronic environment are observed for the residues that either come into direct contact with RNA or that are involved in major conformational changes upon binding to RNA.

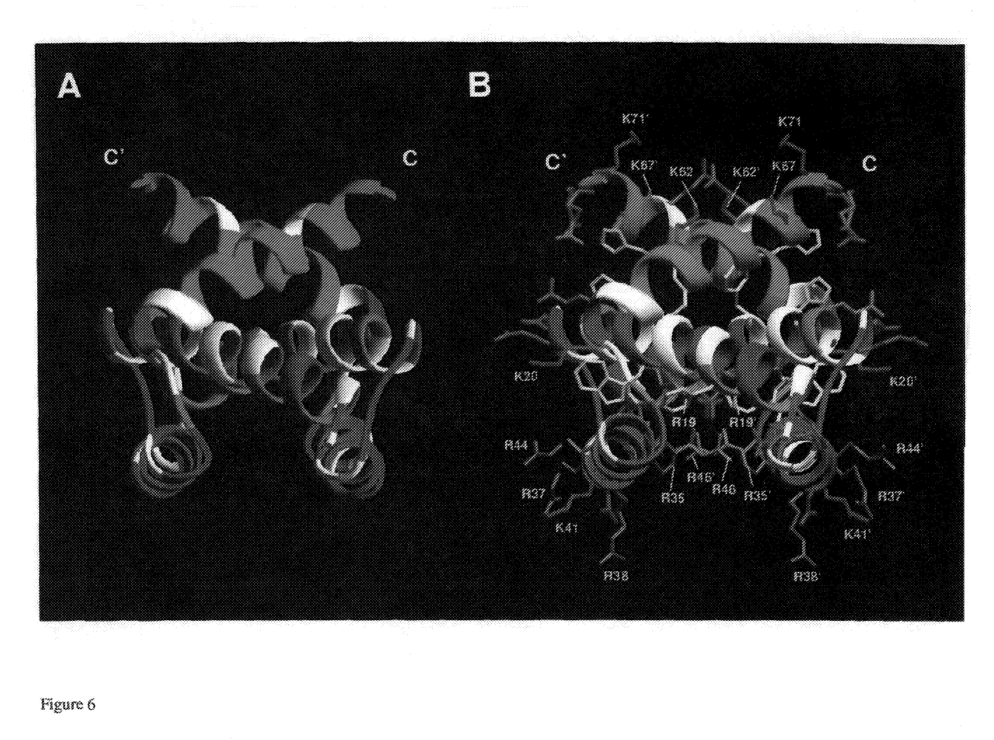

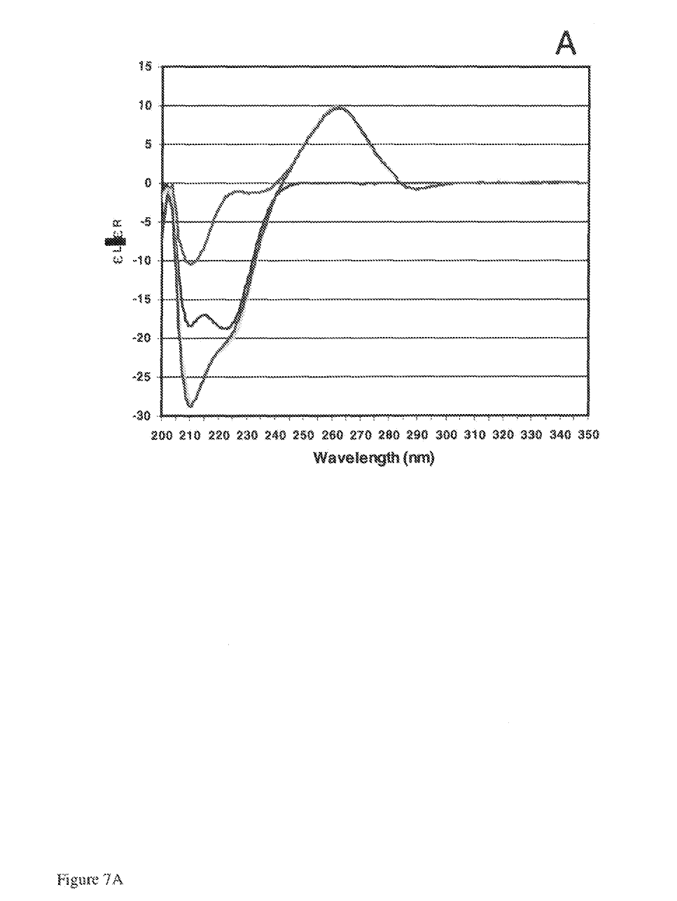

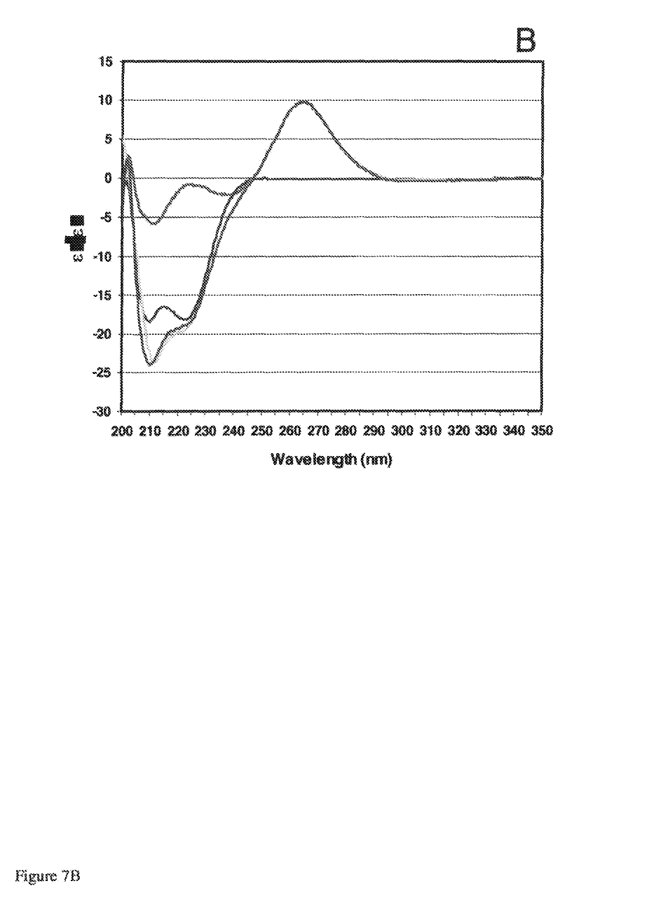

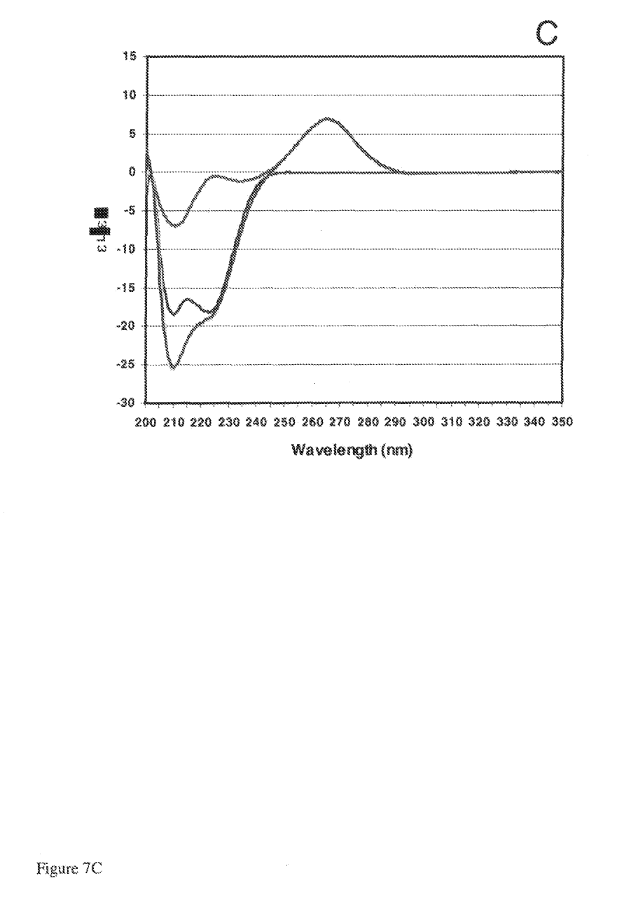

Four HSQC spectra were recorded on samples containing 0.1 mM dimer concentration of NS1A(1-73) with the decreasing molar ratios of dimeric protein to dsRNA as 2:1, 1:1, 1:1.5, and 1:2. Protein was induced to precipitate when this ratio reached above 5:1. In the HSQC spectrum of the 2:1 ratio sample, cross peaks are very broad and difficult to analyze, suggesting that the protein may form larger molecular weight complexes with dsRNA. The spectra with equal or less than 1:1 stoichiometry exhibited only one set of peaks, in spite of the improvement in sensitivity when more dsRNA was introduced. Due to the large size of the NS1A(1-73)-dsRNA complex, de novo backbone assignments for NS1A(1-73) in the complex were not completed. However, by comparison of HSQC spectra for free and dsRNA-bound NS1A(1-73) (FIG. 5B and data generated in the titration experiments described above), it was observed that while no backbone-amide chemical shifts in helices 3 and 3′ were affected by complex formation, almost all residues in helices 2 and 2′ showed ¹⁵N and ¹H shift perturbations upon complex formation. In addition, several residues in helix 1 and 1′ also exhibited chemical shift perturbations upon complex formation. Changes in ¹⁵N and ¹H chemical shifts upon binding were mapped onto the three-dimensional structure of free NS1A(1-73) in FIG. 6. All of the significant chemical shift perturbations observed upon complex formation (represented in cyan) corresponded to NS1A(1-73) backbone atoms that are either in helices 2 and 2′, which contain numerous arginines and lysines, or in helices 1 and 1′ which have close contact with helices 2 and 2′ (FIG. 7B). However, residues whose backbone NHs did not undergo significant chemical shift change, indicative of little or no structural alteration (represented in pink), tended to be distant from the apparent binding epitope. These results confirmed the identification of the ds-RNA binding epitope in regions in or around antiparallel-helices 2 and 2′, as indicated previously by site-directed mutagenesis studies (Wang et al., (1999) RNA, 5:195-205), and further indicated that, as the chemical shifts of amides distant from the binding epitope were not perturbed by complex formation, the overall structure of NS1A(1-73) was not severely distorted by dsRNA-binding.

### Example 17

CIRCULAR DICHROISM (CD) SPECTROSCOPY: Circular dichroism provides a useful probe of the secondary structural elements and global conformational properties of nucleic acids and proteins. For proteins, the 180 to 240 nm region of the CD spectrum mainly reflects the class of backbone conformations (Johnson, W. C., Jr. (1990) Proteins 7:205-214). Changes in the CD spectrum observed above 250 nm upon forming protein-nucleic acid complexes arise primarily from changes in the nucleic acid secondary structure (Gray, D. M. (1996) Circular Dichroism and the Conformational Analysis of Biomolecules, Plenum Press, New York, 469-501). The CD profiles of the four 16 by duplexes (RR, RD, DR, and DD) are distinct and characteristic of their respective duplex types (FIG. 7, red traces). (Gray and Ratliff (1975) Biopolymers 14:487-498; Wells and Yang (1974) Biochemistry 13:1317-1321; Gray et al., (1978) Nucleic Acids Res. 5:3679-3695.) The RR duplex featured a slight negative band at 295 nm, strong negative band at 210 nm, and a positive band near 260 nm, characteristic of the A-form dsRNA conformation (FIG. 7A) (Hung et al., (1994) Nucleic Acids Res. 22:4326-4334; Clark et al., (1997) Nucleic Acids Res. 25:4098-4105). The DD duplex had roughly equal positive and negative bands above 220 nm, with a crossover resulting in a positive band at 260 nm typical of the B-DNA (FIG. 7D) (Id., Gray et al., (1992) Methods Enzymol. 211:389-406). The two hybrids, RD and DR, exhibited traits that were distinct from each other, yet both were roughly intermediate between A-form dsRNA and B-form dsDNA structures (FIGS. 7B, C) ((Hung et al., (1994), Nucleic Acids Res. 22:4326-4334); Roberts and Crothers (1992) Science 258:1463-1466; Ratmeyer et al., (1994) Biochemistry 33:5298-5304; Lesnik and Freier (1995) Biochemistry 34:10807-10815); Clark et al., (1997) Nucleic Acids Res. 25:4098-4105). In addition, the intensity of the positive band at 260 nm appeared most sensitive to the A-like character of the hybird duplex (Clark et al., (1997) Nucleic Acids Res. 25:4098-4105.) CD spectra of NS1A(1-73) in the presence of an equimolar amount of RR, RD, DR, or DD duplex are shown in FIG. 7 (orange traces).

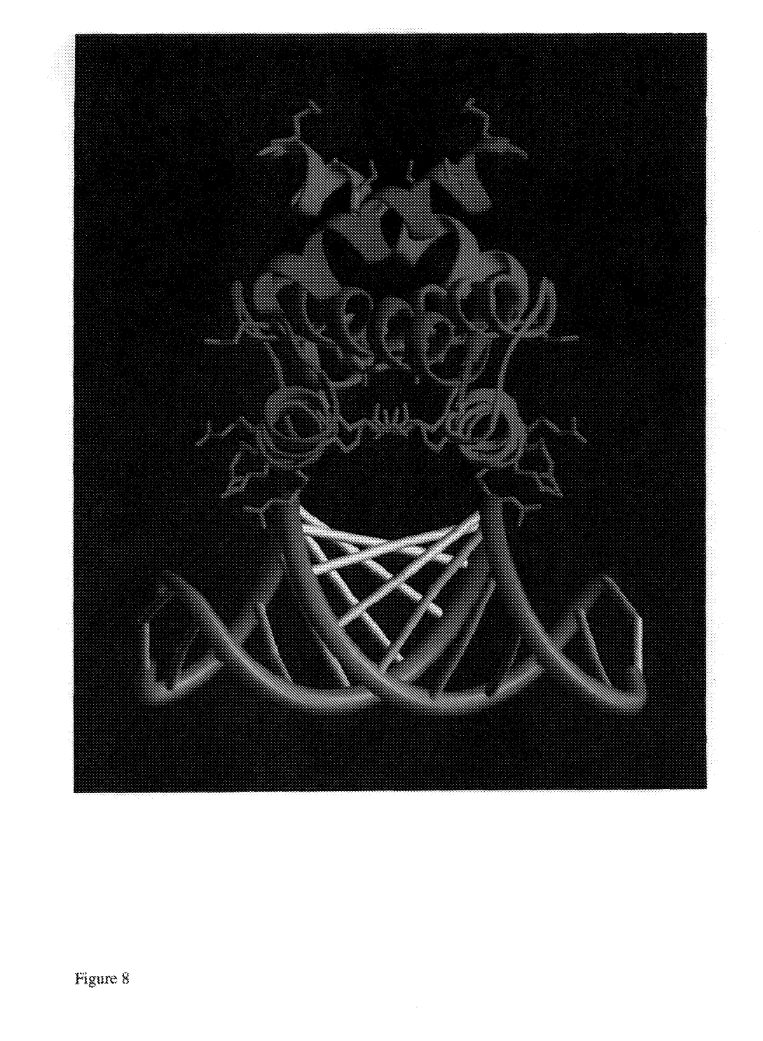

In the dsRNA case (FIG. 7A), gel-filtration purified NS1A(1-73)-dsRNA complex was used to avoid interference due to the presence of free dsRNA (see FIGS. 2 and 3). In each case, the spectrum of free NS1A(1-73) was also shown (blue traces). NS1A(1-73) dominated the CD spectra in the 200-240 nm range (Qian et al., (1995) RNA 1:948-956), while structural information for the nucleic acid duplexes dominated the 250-320 nm region. The gel shift assay and gel filtration data described above showed that only the dsRNA substrate formed a complex with NS1A(1-73). However, as shown in FIG. 8A, complex formation (yellow trace) did not result in significant changes to the 250-320 nm region of the CD spectrum that was most sensitive to nucleic acid duplex conformation. These data demonstrated that the RNA duplex generally retains its A-form conformation in the protein-dsRNA complex. Furthermore, the CD spectrum of the dsRNA-NS1A(1-73) (yellow) and a spectrum computed by simply adding the spectra of free NS1A(1-73) and free dsRNA (green) were also quite similar in the 200-240 nm region, indicating the NS1A(1-73) backbone structure was also not extensively altered by complex formation. Although NS1A(1-73) did not bind to the other duplexes, the CD spectra for each RD, DR, and DD mixed with an equimolar amount of NS1A(1-73) were obtained as controls (FIGS. 7B, C, D). These data confirmed that the detected CD spectra of these mixtures were equal to the sum of separate duplex and protein spectra when the structures of these molecules were not changed.

From the interaction of the N-terminal domain of the NS1 protein from influenza A virus with a 16-bp dsRNA formed from two synthetic oligonucleotides it was established that i) NS1A(1-73) binds to dsRNA, but not to dsDNA or the corresponding hetero duplexes; ii) NS1A(1-73)-dsRNA complex exhibits 1:1 stoichiometry and dissociation constant of ˜1 μmolar; iii) symmetry-related antiparallel helices 2 and 2″ play a central role in binding the dsRNA target; iv) the structures of the dsRNA and the NS1A(1-73) backbone structure are not significantly different in their complex form than they are in the corresponding unbound molecules. Overall, this information provides important biophysical evidence for a working hypothetical model of the complex between this novel dsRNA binding motif and duplex RNA. In addition, this information established that the complex between NS1A(1-73) and the 16 by dsRNA is a suitable reagent for future three-dimensional structural analysis, namely, that it is a homogeneous 1:1 complex.

### Example 18

BIOPHYSICAL CHARACTERIZATION OF THE NS1A(1-73):DSRNA COMPLEX: Gel shift polyacrylamide gel electrophoresis, gel filtration chromatography, and CD spectropolarimetry all demonstrated that NS1A(1-73) bound exclusively to dsRNA and did not exhibit detectable affinity for isosequential dsDNA and hybrid duplexes. A wide body of spectroscopic evidence in the literature, including NMR, Xray, CD, and Raman spectroscopic studies, has established that dsDNA is characterized by a B-type conformation with C2′-endo sugar puckering, dsRNA adopts an A-form structure featuring C3′-endo sugars, and DNA/RNA hybrids exhibit an intermediate conformation between the A- and B-motifs (Hung et al., (1994) Nucleic Acids Res. 22:4326-4334; Lesnik and Freier (1995) Biochemistry 34:10807-10815; Dickerson et al., (1982) Science 216:75-85; Chou et al., (1989) Biochemistry 28:2435-2443; Lane et al., (1991) Biochem. J. 279:269-81; Arnott et al., (1968) Nature 220:561-564; Egli et al., (1993) Biochemistry 32:3221-3237; Benevides et al., (1986) Biochemistry 25:41-50; Gyi et al., (1996), Biochemistry 35:12538-12548; Nishizaki et al., (1996) Biochemistry 35:4016-4025; Salazar et al., (1996) Biochemistry 35:8126-8135; Rice and Gao (1997) Biochemistry 36:399-411; Hashem et al., (1998) Biochemistry 37:61-72; Gray et al., (1995) Methods Enzymol. 246:19-34).

In addition, the topologies of canonical duplexes differ, with the A-form featuring a wide, shallow minor groove while the B-form is characterized by a narrow, deep major groove. Since NS1A(1-73) clearly binds only to dsRNA, yet without sequence specificity, it is clear that this protein discriminates between these nucleic acid helices largely on the basis of duplex conformation (i.e., A-form conformation). However, it cannot be excluded that the molecular recognition process also depends on the presence of 2′-OH groups on each strand of the duplex. These results provide an explanation for the binding of full-length NS1A protein and NS1A(1-73) to another RNA target, a specific stem-bulge in one of the spliceosomal small nuclear RNAs, U6 snRNA (Qian et al, (1994) J. Virol. 68:2433-2441; Wang and Krug, (1996) Virology 223:41-50). It is postulated that this stem-bulge of U6 snRNA forms an A-form structure like dsRNA in solution, allowing NS1A(1-73) to form a complex with U6 snRNA similar to that characterized in this work between NS1A(1-73) and the 16-bp dsRNA fragment.

The sedimentation equilibrium experiments described above established that NS1A(1-73) dimer binds dsRNA duplex in a 1:1 fashion with a dissociation constant, Kd, of ≈1 μM. Interestingly, about 30% of the dsRNA was uncomplexed in size exclusion experiments on 1:1 molar ratios of dimer to duplex (FIG. 2A), and even more free dsRNA was detected in the gel shift assays (FIG. 1). The fraction of unbound dsRNA was found to vary from one NS1A(1-73) preparation to another, and was not observed in gel filtration chromatograms of freshly purified samples of the complex (FIG. 3A). Moreover, it was observed that complexes slowly dissociated during prolonged storage (FIG. 3B). Therefore, it was hypothesized that NS1A(1-73) exhibits slow irreversible self-aggregation under the conditions used in these studies. This hypothesis was also supported by the observation of larger molecules in the sedimentation equilibrium experiments when using laser light scattering as the method of detection. In addition, in some of the gel filtration runs of free NS1A(1-73) samples, a leading peak was observed before the elution of NS1A(1-73) dimer, indicating the possible aggregation. However, when purified NS1A(1-73)-dsRNA complex was reloaded to the gel filtration column, no excessive free dsRNA was observed. The sample behaves like a tight complex with Kd in μM range, consistent with the estimation from sedimentation equilibrium experiments. Complex formation itself, in a way, provided a purification mechanism to isolate the active NS1A(1-73) dimer-active dsRNA complex from “inactive material” present in the sample. Therefore, regardless of the nature of the contaminants, aggregates and/or incompetent species, none of such factors should affect the estimations of the stoichiometry and the dissociation constant based on sedimentation equilibrium experiments using purified NS1A(1-73)-dsRNA complex. Further, the demonstration that the gel purified complexes behave as tight, homogeneous complexes indicated that these complexes are amenable to structural analysis by X-ray crystallography or NMR.

### Example 19

COMPARISON WITH ALTERNATE ESTIMATES OF NS1A(1-73):DSRNA AFFINITY AND STOICHIOMETRY: Previous estimates of NS1A(1-73):dsRNA affinities using gel shift measurements have reported values of apparent dissociation constants (KD) ranging from 20-200 nM (Qian et al., 1995; (Wang et al., 1999). These studies were all carried out with small quantities of longer dsRNA substrates that have different sequences than the substrate used in the biophysical measurements described above. In this earlier work, it was observed that the stoichiometry of NS1A(1-73):dsRNA binding (based on the size of gel shifts) depends on the length of the dsRNA substrate, and that the binding is semi-cooperative (Wang et al., 1999). Similar semi-cooperative binding results have been reported for full length NS1A (Lu et al., (1995) Virology 214, 222-228). The complex between NS1A(1-73) and a 16-bp dsRNA duplex molecule described in this application is a model of part of the complete set of interactions which occur when multiple NS1A RNA-binding domains bind along a longer length of dsRNA, as is thought to occur in vivo. The 1:1 stoichiometry observed in Applicants invention precludes the possible protein-protein interactions and other cooperative effects, which can occur in a multiple-binding mode of a larger system. In the binding of the NS1A protein to larger dsRNAs, the apparent affinity is modulated by configurational entropy effects when there are many possible sites for non-specific binding (Wang et al., (1999) RNA 5, 195-205. For example, Wang et al (1999) have reported that NS1A(1-73) has a 10-fold higher affinity for a 140-bp dsRNA substrate than for a similar 55-bp dsRNA substrate. For these several reasons, the affinity constant reported in the present application for the simple 1:1 complex of NS1A(1-73) dimer with a 16-bp segment of dsRNA is lower than the apparent affinities reported previously for larger cooperative systems. However, while the model complex described in this work captures only part of the full structural information of the complete multiple-binding cooperative system, the complex described in this work is well-characterized, easily generated, and more suitable for detailed structural studies of the protein-dsRNA interactions underlying the NS1A-RNA molecular recognition process.

### Example 20

RNA-BINDING SITE OF NS1A(1-73): Recent alanine scanning mutagenesis studies on NS1A(1-73) (Wang et al., 1999) revealed that binding to larger dsRNA fragments as well as U6 snRNA established that i) the protein must be a dimer in order to bind its target; and ii) only R³⁸ is absolutely required for RNA binding, though K⁴¹ also plays a significant role. The RNA-binding epitope of NS1A(1-73) identified by chemical shift perturbation of 15N-1H HSQC resonances described above supports and extends these mutagenesis data. The chemical shifts of practically all of the backbone amide resonances within helix 2 and 2′ were altered upon binding to the dsRNA. This is consistent with a model in which one or more of the solvent-exposed basic side chains of the residues in helices 2 and 2′, including Arg³⁸ and Lys⁴¹ (FIG. 6B) are involved in the direct contact with dsRNA. It is also possible that the solvent-exposed basic side chains of Arg³⁷ and Arg⁴⁴, as well as the partially buried side chains of Arg³⁵ and Arg⁴⁶ (which participate in intra and intermolecular salt bridges (Chien et al., (1997), Nature Struct. Biol. 4:891-895; Liu et al., (1997) Nature Struct. Biol. 4:896-89917) also interact with dsRNA directly. Moreover, the chemical shift perturbation data also rule out the involvement of the proposed potential RNA binding site on helices 3 and 3′ (Chien et al., (1997)), since most of the backbone 1HN, 15N atoms of residues on the third helix did not show any change in chemical shift upon complex formation, indicating that the binding epitope is distant from helices 3 and 3′ and that the overall backbone conformation of NS1A(1-73) is not affected by RNA binding. Chemical shift differences for some residues on helices 1 and 1′ in the protein core region can be ascribed to the local environment changes induced by the RNA interaction. Overall, these NMR data indicate that the six-helical chain fold conformation of NS1A(1-73) remains intact while binding to dsRNA. This conclusion is in good agreement with the conclusion from CD studies that neither NS1A(1-73) nor dsRNA exhibit extensive backbone structural changes upon complex formation.

### Example 21

A 3D MODEL OF NS1A(1-73)-DSRNA COMPLEX: Analysis of all the data presented here for the NS1A(1-73)-dsRNA complex revealed novel structural features which encode non-specific dsRNA binding functions. The binding site of NS1A(1-73) consists of antiparallel helices 2 and 2′ with an Arg-rich surface. A hypothetical model that is consistent with our cumulative knowledge of the dsRNA binding properties of NS1A(1-73) features a symmetric structure with the binding surface of the protein spanning the minor groove of canonical A-form RNA (FIG. 8). In this hypothetical model outward-directed arginine and lysine side chains of antiparallel helices 2 and 2′ interact in a symmetric fashion with the antiparallel phosphate backbones that form the edges of the major groove, while the surface ion pairs between helices 2 and 2′ form hydrogen-bonded interactions with bases in the minor groove. The strikingly similar spacing between the axes of the 2 and 2′ helices of NS1A(1-73) (˜16.5 Å) and the interphosphate distance across the minor groove (˜16.8 Å) adds further credence to a model in which NS1A(1-73) ‘sits over’ the minor groove of A-form RNA, and requiring A-form conformation for proper docking. Moreover, these protein-RNA interactions require little or no sequence specificity, also consistent with the lack of characterized sequence-specificity in interactions of NS1A with dsRNA (Hatada and Fukuda (1992) J. Gen. Virol. 73, 3325-3329; Lu et al., (1995) Virology 214, 222-228; Qian et al., (1995) RNA 1, 948-956.)

### Example 22

COMPARISON WITH OTHER PROTEIN:DSRNA COMPLEXES: When placed in the context of known RNA-protein interactions, the putative NS1A(1-73):dsRNA model claimed by this application constitutes a novel mode of protein-dsRNA complex formation. Arginine-rich α-helical peptides, such as that derived from the HIV-1 Rev protein, are known to bind dsRNA through specific interactions in the major groove (Battiste et al., (1996), Science 273:1547-1551.) However, the major groove in canonical A-form duplexes is too narrow and deep to accommodate even a single α-helix. As a result, in the Rev-protein-RNA complex binding of the Arg-rich helix results in severe distortions to the structure of the nucleic acid. Id. Hence, an analogous interaction between helices 2/2″ of NS1A(1-73) and the major groove of its dsRNA target can be ruled out since both the protein and nucleic acid retain their free-state conformations upon complex formation. The vast majority of dsRNA-binding proteins typically contain more than one copy of a ubiquitous ca. 70 amino acid, α₁-β₁-β₂-β₃-α₂ module called the dsRNA binding domain (dsRBD) (Fierro-Monti & Matthews, 2000). The X-ray crystal structure of an dsRBD from Xenopus laevis RNA-binding protein A in complex with dsRNA revealed that the two α-helices plus a loop between two of the strands form interactions collectively spanning a 16-bp window—two minor grooves and the intervening major groove—on one face of the duplex (Ryter & Schultz, 1998). Practically all of these protein-RNA contacts involve 2′-OH moieties in the minor groove and non-bridging oxygens in the phosphodiester backbone. A similar view has been recently reported in the NMR structure of a complex between a dsRBD from Drosophila staufen protein and dsRNA (Ramos et al., 2000). As is the case for NS1A(1-73), the protein-dsRNA interactions in both systems are largely non-sequence specific and result in relatively minor perturbations to the structures of both the duplex and free protein (Kharrat et al., 1995; Bycroft et al., 1995; Nanduri et al., 1998). However, unlike the present model, non-helical regions of dsRDB form critical contacts with the nucleic acid. In addition to including non-helical conformations which are essentially for nucleic acid recognition, which are not present in NS1A(1-73) and do not appear to form in NS1A(1-73) upon complex formation, these dsRBM modules lack the symmetry features of NS1A(1-73) which are probably exploited in the molecular recognition process.

### INDUSTRIAL APPLICABILITY

The invention has applications in control of influenza virus growth, influenza virus chemistry, and antiviral therapy.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

All publications cited in the specification are indicative of the level of skill of those skilled in the art to which this invention pertains. All these publications are herein incorporated by reference to the same extent as if each individual publication were specifically and individually indicated to be incorporated by reference.

1-42. (canceled)

43. A method of identifying compounds having inhibitory activity against an influenza virus, comprising:
a) preparing a reaction system comprising an NS1 protein of an influenza virus or a dsRNA binding domain thereof, a dsRNA that binds said protein or binding domain thereof, and a candidate compound; and
b) detecting extent of binding between the NS1 protein and the dsRNA by fluorescence polarization anisotropy, wherein
reduced binding between the NS1 protein and the dsRNA in the presence of the compound relative to a control is indicative of inhibitory activity of the compound against the influenza virus.

44. The method of claim 43, wherein the NS1 protein or dsRNA binding domain thereof is immobilized on a solid support.

45. The method of claim 43, wherein the candidate compound is added to the reaction system prior to or simultaneously with the NS1 protein and the dsRNA.

46. The method of claim 43, wherein the candidate compound is added to the reaction system subsequent to addition of the NS1 protein and the dsRNA.

47. The method of claim 43, further comprising labeling the dsRNA, NS1 protein or dsRNA binding domain thereof with a detectable label, prior to said detecting.

48. The method of claim 43, wherein the NS1 protein or dsRNA binding fragment thereof is present in the reaction system as a fusion protein with glutathione-S-transferase.

49. The method of claim 43, wherein said NS1 protein is a NS1 A protein.

50. The method of claim 43, wherein said NS1 protein is a NS1 B protein.

51. The method of claim 43, wherein the reaction system comprises a fragment of the NS1 protein comprising a dsRNA binding domain of said NS1 protein.

52. The method of claim 51, wherein the dsRNA binding domain comprises NS1A (1-73).

53. The method of claim 51, wherein the dsRNA binding domain comprises NS1B (1-93).

54. The method of claim 43, wherein the method of identification comprises a high throughput screening assay.

55. The method of claim 43, wherein said dsRNA is about 16 by long.

56. A method of identifying compounds having inhibitory activity against an influenza virus, comprising:
a) preparing a reaction system comprising an NS1 protein of an influenza virus or a dsRNA binding domain thereof, a dsRNA that binds said protein or binding domain thereof, and a candidate compound;
b) detecting extent of binding between the NS1 protein and the dsRNA by fluorescence polarization anisotropy, wherein reduced binding between the NS1 protein and the dsRNA in the presence of the compound relative to a control is indicative of inhibitory activity of the compound against the influenza virus; and
c) determining extent of a compound identified in b) as having inhibitory activity to inhibit growth of influenza virus in vitro.

57. The method of claim 56, further comprising d) determining extent of a compound identified in c) as inhibiting growth of influenza virus in vitro, to inhibit replication of influenza virus in a non-human animal.

58. The method of claim 56 wherein said dsRNA is about 16 by long.

59. A method of identifying a compound for use as an inhibitor of influenza virus comprising:
(a) obtaining coordinates for a three-dimensional structure of the influenza virus NS1 protein;
(b) selecting a potential compound by performing rational drug design with said coordinates for a tridimensional structure obtained in step (a), wherein said selecting is performed in conjunction with computer modeling of an NS1-dsRNA complex;
(c) contacting the potential compound with a influenza virus; and
(d) measuring the activity of the influenza virus; wherein a potential compound is identified as a compound that inhibits influenza virus when there is a decrease in the activity of the influenza virus in the presence of the compound relative to in its absence.

60. The method of claim 59, wherein the NS1 protein is a NS1A protein or a dsRNA binding domain thereof.

61. The method of claim 60, wherein dsRNA binding domain is NS1A (1-73).

62. The method of claim 59, wherein the NS1 protein is a NS1B protein or a dsRNA binding domain thereof.

63. The method of claim 62 wherein dsRNA binding domain is NS1B (1-93).


SEQUENCE LISTING (23 sequences)

SEQ ID NO: 1 - protein, 230 aa, Influenza A virus
MDPNTVSSFQVDCFLWHVRKRVADQELGDAPFLDRLRRDQKSLRGRGSTLGLDIETATRAGKQIVERILKEESDEALKMTMASVPASRYLTDMTLEEMSREWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 2 - protein, 230 aa, Influenza A virus
MDSNTITSFQVDCYLWHIRKLLSMSDMCDAPFDDRLRRDQKALKGRGSTLGLDLRVATMEGKKIVEDILKSETNENLKIAIASSPAPRYVTDMSIEEMSREWYMLMPRQKITGGLMVKMDQAIMDKRIILKANFSVLFDQLETLVSLRAFTESGAIVAEISPIPSVPGHSTEDVKNAIGILIGGLEWNDNSIRASENIQRFAWGIRDENGGPSLPPKQKRYMAKRVESEV

SEQ ID NO: 3 - protein, 173 aa, Influenza A virus
ELGDAPFLDRLRRDQKSLKGRGSTLGLNIETATCVGKQIVERILKEESDEAFKMTMASALASRYLTDMTIEEMSRDWFMLMPKQKVAGPLCVRMDQAIMDKNIILKANFSVIFDRLETLTLLRAFTEEGAIVGEISPLPSLPGHTNEDVKNAIGVLIGGLEWNDNTVRVSETL

SEQ ID NO: 4 - protein, 230 aa, Influenza A virus
MDSNTVSSFQVDCFLWHVRKRFADQEMGDAPFLDRLRRDQKSLGGRGSTLGLDIETATRAGKQIVEPILEEESDEALKMTIASAPVSRYLPDMTLEEMSRDWFMLMPKQKVAGSLCIRMDQAIMDKNITLKANFSIIFDRLETLILLRAFTEEGAIVGEISPVPSLPGHTDEDVKNAIGVLIGGLEWNDNTVRDSETLQRFAWRSSNEDRRPPLPPKQKRKMARTIESEV

SEQ ID NO: 5 - protein, 230 aa, Influenza A virus
MDSNTVSSFQVDCFLWHVRKRFADQERGDAPFLDRLRRDQKSLRGRGSTLGLDIETATCAGKQIVERILKEESDEALKMTIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGVLIGGLEWNDNTVRVSETLQRFAWRSSNEDGRPPFPPKQKRKMARTIESEV

SEQ ID NO: 6 - protein, 230 aa, Influenza A virus
MDSNTVSSFQVDCFLWHVRKRFADQKLGDAPFLDRLRRDQKSLRGRASTLGLDIETATRAGKQIVERILEEESNEALKMTIASVPASRYLTDMTLEEMSRDWFMLMPKQKVAGSLCIRMDQAIMEKSIILKANFSVIFDRLETLILLRAFTEEGAIVGEISPLHSLPGHTDEDVKNAVGVLIGGLEWNGNTVRVSENLQRFAWRSRNENERPSLPPKQKREVAGTIRSEV

SEQ ID NO: 7 - protein, 227 aa, Influenza A virus
NTVSSFQVDCFLWHVRKRFADQELGDAPFLDRLRRDQKSLRGRGSTLGLDIETATRAGKQIVERILVEESDEALKMTIVSMPASRYLTDMTLEEMSRDWFMLMPKQKVAGSLCIRMDQAIMDKNIILKANFSVISDRLETLILLRAFTEEGAIVGEISPLPSLPGHTDEDVKNAIGDLIGGLEWNDNTVRVSETLQRFAWRSSNEDGRPLLPPKQKRKMARTIESEV

SEQ ID NO: 8 - protein, 217 aa, Influenza A virus
MDPNTVSSFQVDCFLWHVRKQVADQELGDAPFLDRLRRDQKSLRGRGSTLGLNIETATRVGKQIVERILKEESDEALKMTMASAPASRYLTDMTIEEMSRDWFMLMPKQKVAGPLCIRMDQAIMDKNIILKANFSVIFDRLETLILLRAFTEAGAIVGEISPLPSLPGHTNEDVKNAIGVLIGGLEWNDNTVRVSKTLQRFAWRSSDENGRPPLTPK

SEQ ID NO: 9 - protein, 227 aa, Influenza A virus
NTVSSFQVDCFLWHVLKRFADQELGDAPFLDRLRRDQKSLRGRGSTLGLDIETATRAGKQIVERILEEESDEALKMNIASVPASRYLTDMTLEEMSRDWFMLMPKQKVAGSLCIRMDQAIMDKNIILKANFSVIFDRLETLILLRAFTEEGAIVGEISPLPSLPGHTDEDVKNAIGILIGGLEWNDNTVRVSETLQRFAWRSSNEDGRPPLPPKQKWKMARTIEPEV

SEQ ID NO: 10 - protein, 227 aa, Influenza A virus
NTVSSFQVDCFLWHVRKRFADLELGDAPFLDRLCRDQKSLRGRSSTLGLDIETATRAGKQIVERILEEESDETLKMTIASAPAFRYPTDMTLEEMSRDWFMLMPKQKVAGSLCIRMDQAIMDKNIILKANFSVIFDRLETLILLRAFTEEGAIVGEISPLPSLPGHTNEDVKNAIGDLIGGLEWNDNTVRVSETLQRFAWRSSNEGGRPPLPPKQKRKMARTIESEV

SEQ ID NO: 11 - protein, 230 aa, Influenza A virus
MDSNTITSFQVDCYLWHIRKLLSMRDMCDAPFDDRLRRDQKALKGRGSTLGLDLRVATMEGKKIVEDILKSETDENLKIAIASSPAPRYITDMSIEEISREWYMLMPRQKITGGLMVKMDQAIMDKRITLKANFSVLFDQLETLVSLRAFTDDGAIVAEISPIPSMPGHSTEDVKNAIGILIGGLEWNDNSIRASENIQRFAWGIRDENGGPPLPPKQKRYMARRVESEV

SEQ ID NO: 12 - protein, 124 aa, Influenza A virus
MDSNTITSFQVDCYLWHIRKLLSMRDMCDAPFDDRLRRDQKALKGRGSTLGLDLRVATMEGKKIVEDILKSETDENLKIAIASSPAPRYITDMSIEEISREWYMLMPRQKITGGLMVRPLWTRG

SEQ ID NO: 13 - protein, 230 aa, Influenza A virus
MDSNTVSSFQVDCFLWHVRKRFADQELGDAPFLDRLRRDQKSLRGRGSTLGLDIRTATREGKHIVERILEEESDEALKMTIASVPASRYLTEMTLEEMSRDWLMLIPKQKVTGPLCIRMDQAVMGKTIILKANFSVIFNRLEALILLRAFTDEGAIVGEISPLPSLPGHTDEDVKNAIGVLIGGLEWNDNTVRVSETLQRFTWRSSDENGRSPLPPKQKRKVERTIEPEV

SEQ ID NO: 14 - protein, 202 aa, Influenza A virus
MDPNTVSSFQVDCFLWHVRKRVADQELGDAPFLDRLRRDQKSLKGRGSTLGLNIETATRVGKQIVERILKEESDEALKMTMASAPASRYLTDMTIEEMSRDWFMLMPKQKVAGPLCIRMDQAIMDKSIILKANFSVIFDRLETLILLRAFTEEGAIVGEISPLPSLPGHTNEDVKNAIGVLIGGLEWNDNTVRVSKTLQRFA

SEQ ID NO: 15 - protein, 281 aa, Influenza B virus
MADNMTTTQIEVGPGATNATINFEAGILECYERFSWQRALDYPGQDRLHRLKRKLESRIKTHNKSEPENKRMSLEERKAIGVKMMKVLLFMDPSAGIEGFEPYCVKNPSTSKCPNYDWTDYPPTPGKYLDDIEEEPENVDHPIEVVLRDMNNKDARQKIKDEVNTQKEGKFRLTIKRDIRNVLSLRVLVNGTFLKHPNGDKSLSTLHRLNAYDQNGGLVAKLVATDDRTVEDEKDGHRILNSLFERFDEGHSKPIRAAETAVGVLSQFGQEHRLSPEEGDN

SEQ ID NO: 16 - protein, 281 aa, Influenza B virus
MADNMTTTQIEVGPGATNATINFEAGILECYERLSWQRALDYPGQDRLNRLKRKLESRIKTHNKSEPESKRMSLEERKAIGVKMMKVLLFMDPSAGIEGFEPYCMKSSSNSNCPKYNWTDYPSTPGRCLDDIEEEPEDVDGPTEIVLRDMNNKDARQKIKEEVNTQKEGKFRLTIKRDIRNVLSLRVLVNGTFLKHPNGYKSLSTLHRLNAYDQSGRLVAKLVATDDLTVEDEEDGHRILNSLFERLNEGHSKPIRAAETAVGVLSQFGQEHRLSPEEGDN

SEQ ID NO: 17 - protein, 281 aa, Influenza B virus
MADNMTTTQIEVGPGATNATINFEAGILECYERLSWQRALDYPGQDRLNRLKRKLESRIKTHNKSEPESKRMSLEERKAIGVKMMKVLLFMNPSAGIEGFEPYCMKNSSNSNCPNCNWTDYPPTSGKCLDDIEEEPENVDDPTEIVLRDMNNKDARQKIKEEVNTQKEGKFRLTIKRDIRNVLSLRVLVNGTFLKHPNGYKSLSTLHRLNAYDQSGRLVAKLVATDDLTVEDEEDGHRILNSLFERFNEGHSKPIRAAETAVGVLSQFGQEHRLSPEEGDN

SEQ ID NO: 18 - protein, 281 aa, Influenza B virus
MADNMTTTQIEVGPGATNATINFEAGILECYERLSSQRALDYPGQDRLNRLKRKLESRIKTHNKSEPESKRMSLEERKAIGVKMMKVLLFMNPSAGIEGFEPYCMKNSSNSNCPNCNWTDYPPTPGKCLDDIEEEPENVDDPTEIVLRDMNNKDARQKIKEEVNTQKEGKFRLTIKRDIRNVLSLRVLVNGTFLKHPNGYKSLSTLHRLNAYDQSGRLVAKLVATDDLTVEDEEDGHRILNSLFERFNEGHSKPIRAAETAVGVLSQFGQEHRLSPEEGDN

SEQ ID NO: 19 - protein, 281 aa, Influenza B virus; identical to SEQ ID NO: 15

SEQ ID NO: 20 - protein, 281 aa, Influenza B virus
MADNMTTTQIEVGPGATNATINFEAGILECYERLSWQRALDYPGQDRLNRLKRKLESRIKTHNKSEPESKRMSLEERKAIGVKMMKVLLFMDPSAGIEGFEPYCMKSSSNSNYPKYNWTDYPSTPGRCLDDIEEETEDVDDPTEIVLRDMNNKDARQKIKEEVNTQKEGKFRLTIKRDIRNVLSLRVLVNGTFLKHPNGYKSLSTLHRLNAYDQSGRLVAKLVATDDLTVEDEEDGHRILNSLFERLNEGHSKPIRAAETAVGVLSQFGQEHRLSPEEGDN

SEQ ID NO: 21 - protein, 281 aa, Influenza B virus
MADNMTTTQIEVGPGATNATINFEAGILECYERLSWQRALDYPGQDRLNRLKRKLESRIKTHNKSEPESKRMSLEERKAIGVKMMKVLLFMDPSAGIEGFEPYCMKSSSNSNYPKYNWTNYPSTPGRCLDDIEEETEDVDDPTEIVLRDMNNKDARQKIKEEVNTQKEGKFRLTIKRDIRNVLSLRVLVNGTFLKHPNGYKSLSTLHRLNAYDQSGRLVAKLVATDDLTVEDEEDGHRILNSLFERLNEGHPKPIRAAETAVGVLSQFGQEHRLSPEEGDN

SEQ ID NO: 22 - protein, 281 aa, Influenza B virus; identical to SEQ ID NO: 16

SEQ ID NO: 23 - protein, 281 aa, Influenza B virus
MADNMTTTQIEVGPGATNATINFEAGILECYERLSWQRALDYPGQDRLNRLKRKLESRIKTHNKSEPESKRMSLEERKAIGVKMMKVLLFMDPSAGIEGFEPYCMKSSSNSNCPKYNWTDYPSTPGRCLDDIEEEPEDVDGPTEIVLRDMNNKDARQKIKEEVNTQKEGKFRLTIKRDIRNVLSLRVLVNGTFLKHPNGYKSLSTLHRLNAYDQSGRLVAKLVATDDLTVEDEEDGHRILNSLFERLNEGHSKPIRAAETAMGVLSQFGQEHRLSPEEGDN